(19) United States
(12) Patent Application Publication
(10) Pub. No.: US 2010/0233327 A1
(43) Pub. Date: Sep. 16, 2010

(54) SYSTEM AND METHOD FOR FORMULATING COMPOSITIONS OF CONCENTRATED LIQUID SWEETENERS FOR INDIVIDUAL SERVINGS IN RECYCLABLE AND COMPOSTABLE PACKAGING

(76) Inventor: Seth J. Hersh, Los Angeles, CA (US)

Correspondence Address: BERRY & ASSOCIATES P.C., 9229 SUNSET BOULEVARD, SUITE 630, LOS ANGELES, CA, 90069, US

(21) Appl. No.: 12/402,488

(22) Filed: Mar. 11, 2009

(51) Int. Cl. A23L 1/221; G05D 11/02; G01N 33/02; B65D 65/40; B65D 85/72

(52) U.S. Cl. 426/122; 426/650; 366/152.2; 426/232; 426/126

(57) ABSTRACT

A system and method for formulating compositions of concentrated liquid sugar that are provided in individual serving sizes in recyclable and compostable packaging, which are configured for ease of use. In accordance with a preferred embodiment of the invention, the liquid sugar composition comprises at the very least a predetermined ratio of a finite amount of natural, organic, pure cane sugar or natural sugar substitute, to a finite amount of water. The composition of liquid sugar in accordance with the preferred embodiment has a brix degree measurement greater than 67.5. The preferred compositions of concentrated liquid sugar are formulated with different ingredients that are configured to sweeten a desired drink uniformly, while dissolving instantly and without diluting it unnecessarily and spoiling its natural taste. Moreover, these preferred compositions of liquid sugar are completely devoid of any taste other than that of natural sugar, nor do they have any discernible color. These preferred compositions of concentrated liquid sugar are formulated by an improved system and method facilitating portions for individual use. In addition, as a result of the increasing recognition of the advantages of a healthier, environmentally-conscious existence, these compositions of concentrated liquid sugar are produced in an environmentally conscious factory and packaged in recyclable and compostable sachets that are configured to facilitate ease of use by individuals.

### BACKGROUND OF THE INVENTION

1. Field of the Invention

The present invention relates generally to compositions of liquid sweeteners. More particularly, the present invention relates to compositions of concentrated liquid sweeteners, such as sugar, formulated and packaged for individual servings in recyclable and compostable packaging that is configured to facilitate easy dispensing of the liquid sweetener by a user. The present invention also provides an improved system and method of formulating concentrated forms of the liquid sweeteners for individual serving sizes. 2. Introduction

Everyone is familiar with the fact that dissolving natural, granulated sugar in a solution invariably results in an uneven distribution of sugar in the solution. In every instance, sugar crystals float to the bottom of a glass resisting dissolution. As a result, the drink that a consumer wants to sweeten is not uniformly sweet, its sweetness greatly intensified towards the last sips. As alternatives to natural granulated sugar, there are many unnatural, man-made, artificial sweetener “derivatives” such as sugar-like dextrose, saccharine, aspartame, maltodextrin, phenylalanine, sucralose and the like, which are harmful for the body, albeit lower in calories.

The best remedy is a liquid sugar syrup, which could be used to sweeten beverages and other drinks. Liquid sugar syrups are typically formulated in large quantities to sweeten bottled soft drinks in factories. Liquid flavored sweeteners are also available in bottles for use by individuals. For example, one such natural liquid sweetener is honey, which however, has a distinctive flavor and color because of the differences between its principal nectar sources, for example, orange blossom or clover. Such distinctive flavors only serve to alter the taste of the drink they are added to. Moreover, the viscosity of honey is a detriment to its ability to dissolve with ease, therefore, not providing a viable solution.

Another liquid sweetener available on the market today, is Agave nectar and liquid Stevia extract. The former has a distinctive sour taste associated with it and only comes packaged in a squeeze bottle. The latter has a bitter and undesirable taste that lingers and is available only in a bottle that utilizes an eye drop application. There are no compositions of liquid sugar that exist today for use by individuals to sweeten their drinks without altering the natural taste of their drinks or beverages.

In addition, many of the liquid flavored syrups or sweeteners available in bottles require refrigeration after opening in order to slow the re-crystallization process of sugar and to maintain the integrity of the product.

By way of example, U.S. Pat. No. 4,193,817 describes a process for production of liquid sugar suitable for use in bottled soft drinks and comparable in color to liquid sugar produced primarily from refined sugar. Various techniques for decolorization of sugar liquors and sugar syrups exist, in production of refined sugar in refineries, conventionally before crystallization from sugar liquors. Such techniques have employed various carbonaceous materials, ionic-exchange resins, ionic-sorption resins, and various other materials. One technique of particular interest has employed chloride form of Type-1 strong-base anion-exchange resin. This technique employing chloride form of Type-1 strong-base anion-exchange resin, as applied variously to sugar liquors and sugar syrups, is described in various publications including U.S. Pat. No. 2,785,998 to F. H. Harding et al.; G. Merrill Andrus, “Sugar Decolorization with Anion-Exchange Resins”, Reprint from the May 1967, issue of Sugar y Azugar; F. X. McGarvey, “The Evaluation of Ion Exchange Resins for Sugar Liquor Decolorization”, Paper presented to Meeting of Sugar Industry Technicians, New York, May 2-4, 1965; and Duolite Ion Exchange Resins in the Treatment of Sugar Solutions. COPYRIGHT 1972 Diamond Shamrock Corporation, particularly at pages 38 through 40. The process described in this patent enables bottler's liquid sugar advantageously and economically to be produced outside sugar refineries, as by a user of bottler's liquid sugar, and is expected to alleviate local shortages of refined sugar.

Moreover, most food items today, whether solids or liquids are stored, packaged, and shipped in packaging materials that protect them from harmful elements, such as gases, moisture, light, microorganisms, leaking, spilling, or the like. Many of these materials are characterized as being disposable, but in fact, they have little recyclable and compostable ability or advantage, much less, biodegradability. As is well known, packaging materials are damaging to our environment and use harmful chemicals that are believed to be one of the causes of the destruction of the ozone layer. Due to heightened environmental awareness today, there is significant pressure to discontinue use of such products that are harmful to our environment and to seek more eco-friendly options.

Accordingly, there exists a need for compositions of concentrated liquid sweeteners that are preferably natural and formulated for individual servings in eco-friendly packaging.

### SUMMARY OF THE INVENTION

The present invention relates to compositions of concentrated liquid sweeteners, such as sugar, that are provided in individual serving sizes in recyclable and compostable packaging that is configured for ease of use. In accordance with a preferred embodiment of the invention, the liquid sugar composition comprises at the very least, a predetermined ratio of a finite amount of natural, organic, pure cane sugar, natural sugar substitute, or the like, to a finite amount of water. The composition of liquid sugar in accordance with the preferred embodiment should have a brix degree measurement greater than 67.5. In accordance with preferred embodiments of the present invention, compositions of the concentrated liquid sweeteners are formulated with different ingredients, variations of which are disclosed in greater detail in the detailed description, which follows below.

These preferred compositions of concentrated liquid sweeteners are configured to sweeten a desired drink uniformly, while dissolving instantly and without diluting it unnecessarily and spoiling its natural taste. Moreover, the improved composition of liquid sugar is completely devoid of any taste other than that of natural sugar, nor does it have any discernable color.

In accordance with yet another embodiment of the present invention, the compositions of concentrated liquid sugar are formulated by an improved system and method facilitating portions for individual use.

In accordance with still another embodiment of the present invention, as a result of the increasing recognition of the advantages of a healthier, environmentally-conscious existence, the compositions of concentrated liquid sugar are produced in an environmentally conscious factory and packaged in recyclable and compostable sachets.

In yet another embodiment of the present invention, the recyclable and compostable sachets are configured to facilitate ease of use and prevent waste of the any liquid sugar compositions.

Additional features and advantages of the invention will be set forth in the description which follows, and in part will be obvious from the description, or may be learned by practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth herein.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof, which are illustrated in the appended drawings. Understanding that these drawings depict only typical embodiments of the invention and are not therefore, to be considered to be limiting of its scope, the invention will be described and explained with additional specificity and detail through the use of the accompanying drawings in which:

FIG. 1 is a front schematic view of a recyclable and compostable packaging or sachet with an individual serving of a composition of liquid sweetener (e.g., sugar) formulated in accordance with one of many embodiments of the present invention;

FIG. 2 is a front schematic view of another recyclable and compostable packaging or sachet with an individual serving of a composition of liquid sweetener (e.g., sugar substitute, preferably natural, but also, artificial) formulated in accordance with one of many embodiments of the present invention;

FIG. 3 is a rear schematic view of the recyclable and/or compostable packaging or sachet shown in FIGS. 1 and 2;

FIG. 4 is a front schematic view of the recyclable and/or compostable packaging or sachet shown in FIGS. 1 and 2 illustrating a perforated edge (along one edge) configured to easily detach with slight pressure by a user and reveal a concealed orifice;

FIG. 5 is a front schematic view of the recyclable and compostable packaging or sachet shown in FIGS. 1 and 2 illustrating a perforated edge (along its entire width) configured to easily detach with slight pressure by a user and reveal a concealed orifice;

FIG. 6 is a front schematic view of the recyclable and compostable packaging or sachet shown in FIGS. 1, 2, and 4, illustrating the perforated edge, slightly detached, revealing the spout-like orifice through which the liquid sugar composition can be easily poured out;

FIG. 6A is a cross sectional view taken along A-A of FIG. 6 illustrating the layer composition of the recyclable and/or compostable packaging;

FIG. 7 is a block diagram illustrating the various components of a system by which the liquid sugar composition in accordance with the many preferred embodiments of the present invention is formulated and packaged in recyclable and/or compostable packaging or sachets with individual serving sizes; and

FIG. 8 is a flow diagram illustrating the process by which the liquid sweetener (e.g., sugar) composition in accordance with the many preferred embodiments of the present invention is formulated and packaged in recyclable and/or compostable packaging or sachets with individual serving sizes.

### DETAILED DESCRIPTION OF THE INVENTION

Various embodiments of the invention are described in detail below. Whereas specific compositions involving many exemplary ingredients are described here, it should be understood that the description here is merely illustrative and not intended to limit the scope of the various aspects of the invention. A person skilled in the relevant art will recognize that other ingredients may be easily used or substituted than those that are described here without parting from the spirit and scope of the invention. In addition, the system and method for formulating the specific compositions that are described here are also merely illustrative and not intended to limit the scope of the inventions that are claimed here.

Before embarking into a detailed description of the present inventions, definitions of certain terms and processes used in the industry and used here are described to allow a reader to fully comprehend the nature and scope of the inventions that are described here. Variations to the embodiments disclosed here may be obvious to those skilled in the art in view of the descriptions here.

### Brix

Degrees Brix (symbol ° Bx) is a measurement of the dissolved sugar-to-water mass ratio of a liquid. It is measured with a saccharimeter that measures specific gravity of a liquid and can also and more easily be measured using a refractometer. A 50° Bx solution is 50% (w/w), with 50 grams of sugar per 100 grams of solution. Or, to describe it another way, there are 50 grams of sucrose sugar and 50 grams of water in the 100 grams of solution.

Brix is commonly used in the food industry for measuring the approximate amount of sugars in fruits, vegetables, juices, wine, and soft drinks. Different countries use the scales in different industries; in the United Kingdom, brewing is measured with specific gravity X 1000, European brewers use Plato degrees, and U.S. industries use a mix of specific gravity, Brix, Baumé and Plato degrees. Because Brix is a measure of sugar by weight in liquid it correlates directly with perceived sweetness. The concentration of dissolved solids (mostly sucrose) in a fluid is therefore related to the specific gravity of that liquid.

The most common of Brix-measuring instruments used today are digital refractometers that calculate the Brix value based on refractive index. These meters are generally portable, splash proof, and very simple to use, so that they can be operated by anyone in any location.

### Viscosity

The study of viscosity is known as rheology. Viscosity as it is used in the present invention (and for liquids only) is a measure of the “thickness” of a fluid. Water, for example is “thin” and therefore, has a lower viscosity. Vegetable oil, on the other hand, is considered to be “thick” and therefore, has a higher viscosity. Viscosity describes a fluid's internal resistance to flow and may be described as a measure of fluid friction. There are various ways to determine and measure viscosity. A viscometer, or a viscosimeter, is an instrument used to measure the viscosity of a fluid, but only under one flow condition. For liquids with viscosities that vary with flow conditions, an instrument called a rheometer can be used.

In general, the measurement is commonly achieved in one of two ways, either the fluid remains stationary and the instrument moves through it, or the instrument is stationary and the fluid moves past it. The drag caused by relative motion of the fluid and a surface is a measure of the viscosity. For example, at 68 degrees Fahrenheit (20 C) the viscosity of water is 1.002 pascal-seconds (Pa s).

Two common instruments for measuring kinematic viscosity are the glass capillary viscometer (typically used to measure the viscosity of paint) and the Ford viscosity cup. The Ford viscosity cup, measures the rate of flow of a liquid which under ideal conditions, is proportional to the kinematic viscosity. Vibrating viscometers, such as the Dynatrol, utilize vibration rather than rotation to measure viscosity.

### Invert Sugar

Inverted sugar syrup can be easily made by adding roughly one gram of citric acid or ascorbic acid, cream of tartar, or fresh lemon juice (10 ml/k) per kilogram of sugar. The mixture is brought to a boil and simmered for roughly 15-20 minutes, and will convert a large majority of the sucrose to effectively prevent crystallization, without yielding a noticeably sour taste. Invert sugar syrup may also be produced without the use of acids or enzymes by thermal means alone: two parts granulated sucrose to one part water simmered for five to seven minutes will invert a modest portion of the sugar. All inverted sugar syrups are created from hydrolysing sucrose to glucose (dextrose) and fructose by heating a sucrose solution with the catalytic properties of an acid or enzyme to aid in conversion. Commercially prepared acid catalyzed solutions are neutralized when the desired level of inversion is reached. Invert sugar has a lower water activity than that of sucrose, therefore, it has a high viscosity and longer shelf life in products that utilize it. The shelf life of partial inverts is approximately six months, depending on storage and climatic conditions. Should a partially inverted syrup develop sugar crystals the solution may be restored to liquid state by gently heating.

There are various types of sweeteners that are available, some examples of which are listed below: cane sugar, brown sugar, beet sugar, fruit sugar, Sweet N' Low (saccharine), Equal (aspartame), Splenda (sucralose), Sweet Simplicity (aspartame), NutraSweet (aspartame), NatraTaste (aspartame), Sugar for the Soul (refined sugar), Agave Nectar, Sugar in the Raw (Turbinado sugar), Stevia (rebiana) and Truvia (erythritol, rebiana). Other artificial sugar substitutes exist, most of which can be found on the market at specialty food stores like Whole Foods. Some of these products are dextrose, isomalt, mannitol, trehalose, sucromalt, maltitol, sorbitol, and xylitol. Product information on any one of these sweeteners is readily available. By way of example, information on liquid sucrose may be provided as indicated below.

Liquid Sucrose
Product Information - The following is typical information that may be of
use in answering questions regarding this ingredient.
Cargill's Liquid Sucrose is a pure 57.5° Brix sucrose solution which
is ideal for use in many food applications.
REPRESENTATIVE CHEMICAL AND PHYSICAL DATA
Typical Analysis

| Brix | 57.6 +/− 0.3° | |
|---|---|---|
| Sulfated Ash (%) | 0.05 | max |
| pH | 7.0-8.0 | |
| Invert (% Dry Basis) | 0.30 | max |
| Sediment | 4 | ppm max |

| | |
|---|---|
| Color | Clear to light straw |

Microbiological Limits

| | |
|---|---|
| Mesophilic Bacteria | 200 cfu/10 g max |
| Yeast | 10 cfu/10 g max |
| Mold | 10 cfu/10 g max |

Sensory Characteristics

| | |
|---|---|
| Appearance | Clear Liquid |
| Taste | Sweet, Bland |
| Odor | Characteristic |

Density and Viscosity Factors

| | | Pounds/ | Pounds/ | |
|---|---|---|---|---|
| Temp | Specific Gravity | Gallon | Gallon | Viscosity |
| (° F.) | (Temp ° F./60° F.) | (Temp ° F.) | (DSB) | cP |
| 70 | 1.3331 | 11.11 | 7.50 | 242 |
| 80 | 1.3301 | 11.09 | 7.48 | 160 |
| 90 | 1.3271 | 11.08 | 7.47 | 112 |
| 100 | 1.3240 | 11.04 | 7.45 | 79 |
| 110 | 1.3207 | 11.01 | 7.43 | 59 |

Regulatory Data/Labeling Data
United States

| | |
|---|---|
| GRAS Affirmation | CFR 21 184.1854 |
| Labeling | Liquid Sucrose/Sugar, Sucrose Syrup |

Canada

| | |
|---|---|
| FDR | B.18.002 |
| Labeling | Liquid Sugar |

Shelf Life and Storage
The recommended storage temperature range for Liquid Sucrose
is 75-100° F. Liquid Sucrose stored for extended periods (over
30 days) should be evaluated periodically for fitness of use.

There are also several liquid sugar products, a few of which are listed here: Frigolet (syrups), Torani (syrups), Monin (syrups), Stirrings (syrups and cocktail mixers), Sugar Shots (liquid sugars), Finest Call (syrups), Depaz Cane Syrup (rum producers), Da Vinci Gourmet (syrup), and Wholesome Sweeteners (Fair Trade Certified, organic and natural sugars, syrups and nectars).

It should be appreciated that the preferred concentrated liquid sugar compositions formulated in accordance with the present inventions contemplate use of natural products from natural sources, rather than artificial sweeteners or high fructose corn syrup. However, in the event such artificial sweeteners are also provided in liquid form in individual serving sized recyclable and compostable packaging, they would fall within the scope of the inventions disclosed here.

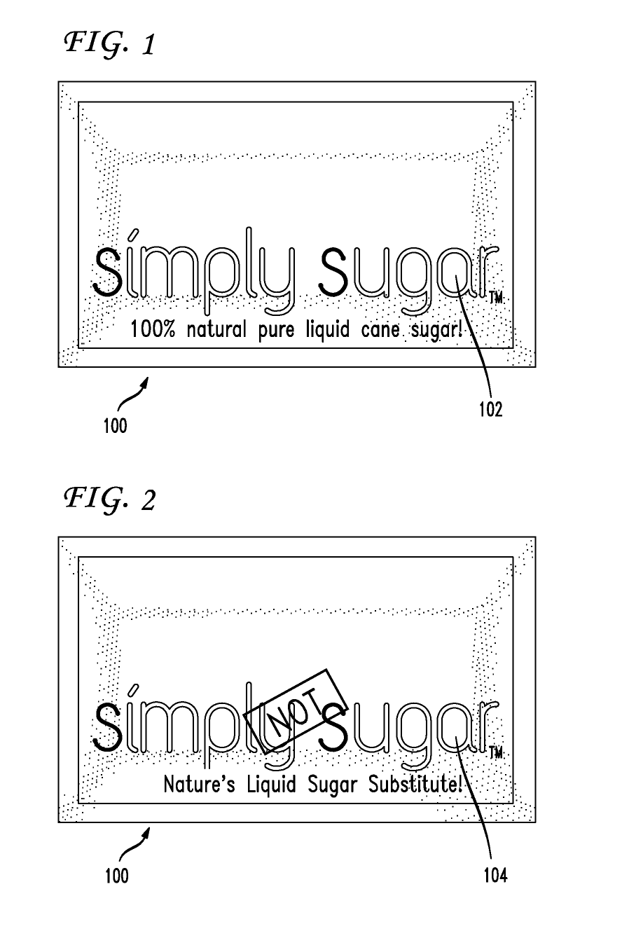

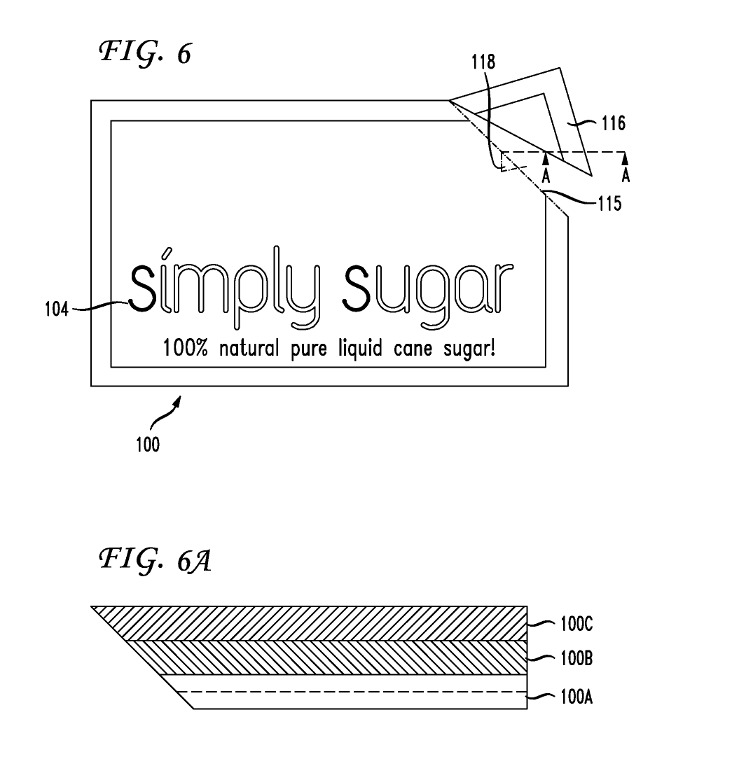

FIG. 1 illustrates a packaging or sachet 100 suitably sized to house an individual serving of a concentrated liquid sweetener (e.g., sugar) composition formulated in accordance with preferred embodiments of the present invention. The packaging or sachet is made from a recyclable and/or compostable material that is not only eco-friendly when it is disposed of by a consumer, but is also manufactured in an eco-friendly environment in accordance with the guidelines for development of environmentally friendly products. Use of such recyclable and/or compostable material reduces environmental pollutants, helps conserve resources and energy, incorporates sustainable resources, supports long-term use, is re-usable, is sometimes bio-degradable, incorporates recycled materials, and above all, is easy to process and dispose of. The sachet 100 is composed of a substrate with at least three separate layers of material. Referring now to FIG. 6A, the first layer is polyethylene terephthalate with polyester (commonly referred to as “PET”) and indicated by reference numeral 100A, which is a healthy alternative to PVC because it has little or no VOC (volatile organic compounds). Therefore, PET is a “green” product. The second layer indicated by reference numeral 100B is commonly referred to as “VMPET,” which is an aluminum vacuum metalized version of PET. To formulate this layer, aluminum is injected into a PET layer to lend it “vacuumed” freshness ability. The third layer indicated by reference numeral 100C is Linear Low density Polyethylene, more commonly referred to as “LLDPE.”. This layer once subjected to heat and pressure, seals the sachet, with the composition inside, resulting in a final product as illustrated in FIGS. 1 and 2.

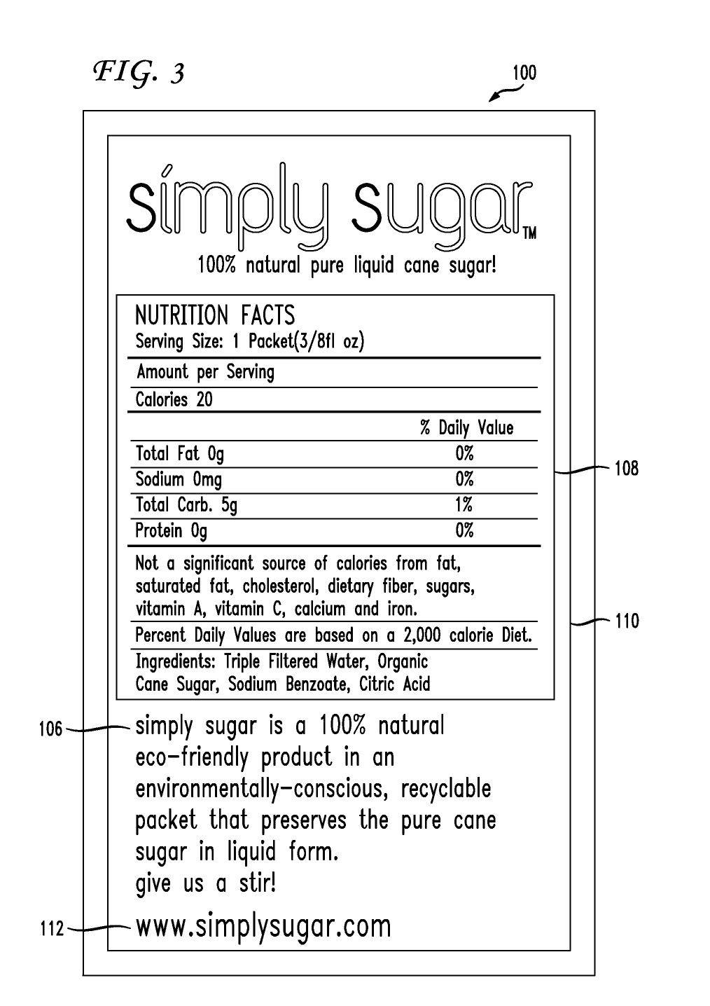

FIGS. 1 and 2 illustrate different visual indicia as indicated by reference numerals 102 and 104 to illustrate if the liquid sugar composition within the sachet 100 is natural sugar or a natural sugar substitute or a composition with artificial ingredients. FIG. 3 illustrates more detailed visual indicia 106 including visual indicia illustrative of the nutrition facts 108 and general descriptive indicia 110 along with website indicia 112. The visual indicia 102, 104, or 106 may be illustrated on a fourth, final layer that is a cosmetic layer, such as a paper layer.

Advantageously, because the sachets 100 are vacuumed sealed and individually portioned, they require no refrigeration, as is typical with many other liquid sugar products packaged in bottles to prolong its shelf life. Without refrigeration, re-crystallization of the sugar content occurs, thereby destroying the integrity of any liquid composition.

Most syrups are formulated with equal parts of sugar and water. Heating the water assists the dissolution of the sugar content. Heating both the sugar and water portions to their respective boiling points ensure that every crystalline particle of sugar is completely dissolved and a portion of the water has evaporated thereby further concentrating the solution. The only test to measure the actual degree of concentration is to utilize a saccharimeter, or more easily with a refractometer, or Baume scale that measures the specific gravity of a liquid. For the purposes of the concentrated liquid sugar composition formulated in accordance with the present invention, a higher ratio of sugar and sugar substitute to water is utilized whereby the resulting composition is sweeter by volume than basic simple syrup. However the viscosity of this composition remains low as to not compromise the uniform sweetening capability and immediate dissolution. It is the specific balance between sugar, water, and temperature that yields this product's uniqueness. A higher brix degree directly correlates with a higher ratio of sugar to water. This gives the composition a more intense sweetness.

The various ingredients that are used to formulate various compositions of natural liquid sugar with a brix degree measurement of greater than 67.5 are listed below in Table 1.

TABLE 1
COMPOSITIONS WITH NATURAL SUGAR

| Composition of Liquid Sugar | Ingredients |
|---|---|
| Composition 1 | Cane Sugar, water |
| Composition 2 | Cane Sugar, water, sodium benzoate, citric acid |
| Composition 3 | Cane Sugar, water, lactic acid |
| Composition 4 | Cane Sugar, mineral water, |
| Composition 5 | Cane Sugar, mineral water, sodium benzoate, citric acid |
| Composition 6 | Cane Sugar, mineral water, lactic acid |
| Composition 7 | Cane Sugar, natural spring water, |
| Composition 8 | Cane Sugar, natural spring water, sodium benzoate, citric acid |
| Composition 9 | Cane Sugar, natural spring water, lactic acid |
| Composition 10 | Cane Sugar, filtered water, |
| Composition 11 | Cane Sugar, filtered water, sodium benzoate, citric |
| Composition 12 | Cane Sugar, filtered water, lactic acid |
| Composition 13 | Cane Sugar, artesian water, |
| Composition 14 | Cane Sugar, artesian water, sodium benzoate, citric acid |
| Composition 15 | Cane Sugar, artesian water, lactic acid |
| Composition 16 | Cane Sugar, purified water, |
| Composition 17 | Cane Sugar, purified water, sodium benzoate, citric acid |
| Composition 18 | Cane Sugar, purified water, lactic acid |
| Composition 19 | Cane Sugar, distilled water, |
| Composition 20 | Cane Sugar, distilled water, sodium benzoate, citric, acid |
| Composition 21 | Cane Sugar, distilled water, lactic acid |
| Composition 22 | Cane Sugar, vapor distilled electrolyte enhanced water, |
| Composition 23 | Cane Sugar, vapor distilled electrolyte enhanced water, sodium |
| | benzoate, citric acid |
| Composition 24 | Cane Sugar, vapor distilled electrolyte enhanced water, lactic |
| | acid |
| Composition 25 | Cane Sugar, triple filtered water, |
| Composition 26 | Cane Sugar, triple filtered water, sodium benzoate, citric acid |
| Composition 27 | Cane Sugar, triple filtered water, lactic acid |
| Composition 28 | Organic cane sugar, water |
| Composition 29 | Organic cane sugar, water, sodium benzoate, citric acid |
| Composition 30 | Organic cane sugar, water, lactic acid |
| Composition 31 | Organic cane sugar, mineral water |
| Composition 32 | Organic cane sugar, mineral water, sodium benzoate, citric |
| | acid |
| Composition 33 | Organic cane sugar, mineral water, lactic acid |
| Composition 34 | Organic cane sugar, natural spring water |
| Composition 35 | Organic cane sugar, natural spring water, sodium benzoate, |
| | citric acid |
| Composition 36 | Organic cane sugar, natural spring water, lactic acid |
| Composition 37 | Organic cane sugar, filtered water |
| Composition 38 | Organic cane sugar, filtered water, sodium benzoate, citric |
| Composition 39 | Organic cane sugar, filtered water, lactic acid |
| Composition 40 | Organic cane sugar, artesian water |
| Composition 41 | Organic cane sugar, artesian water, sodium benzoate, citric |
| | acid |
| Composition 42 | Organic cane sugar, artesian water, lactic acid |
| Composition 43 | Organic cane sugar, purified water |
| Composition 44 | Organic cane sugar, purified water, sodium benzoate, citric |
| | acid |
| Composition 45 | Organic cane sugar, purified water, lactic acid |
| Composition 46 | Organic cane sugar, distilled water |
| Composition 47 | Organic cane sugar, distilled water, sodium benzoate, citric, |
| | acid |
| Composition 48 | Organic cane sugar, distilled water, lactic acid |
| Composition 49 | Organic cane sugar, vapor distilled electrolyte enhanced water |
| Composition 50 | Organic cane sugar, vapor distilled electrolyte enhanced water, |
| | sodium benzoate, citric acid |
| Composition 51 | Organic cane sugar, vapor distilled electrolyte enhanced water, |
| | lactic acid |
| Composition 52 | Organic cane sugar, triple filtered water |
| Composition 53 | Organic cane sugar, triple filtered water, sodium benzoate, |
| | citric acid |
| Composition 54 | Organic cane sugar, triple filtered water, lactic acid |
| Composition 55 | Beet sugar, water |
| Composition 56 | Beet sugar, water, sodium benzoate, citric acid |
| Composition 57 | Beet sugar, water, lactic acid |
| Composition 58 | Beet sugar, mineral water |
| Composition 59 | Beet sugar, mineral water, sodium benzoate, citric acid |
| Composition 60 | Beet sugar, mineral water, lactic acid |
| Composition 61 | Beet sugar, natural spring water |
| Composition 62 | Beet sugar, natural spring water, sodium benzoate, citric acid |
| Composition 63 | Beet sugar, natural spring water, lactic acid |
| Composition 64 | Beet sugar, filtered water |
| Composition 65 | Beet sugar, filtered water, sodium benzoate, citric |
| Composition 66 | Beet sugar, filtered water, lactic acid |
| Composition 67 | Beet sugar, artesian water |
| Composition 68 | Beet sugar, artesian water, sodium benzoate, citric acid |
| Composition 69 | Beet sugar, artesian water, lactic acid |
| Composition 70 | Beet sugar, purified water |
| Composition 71 | Beet sugar, purified water, sodium benzoate, citric acid |
| Composition 72 | Beet sugar, purified water, lactic acid |
| Composition 73 | Beet sugar, distilled water |
| Composition 74 | Beet sugar, distilled water, sodium benzoate, citric, acid |
| Composition 75 | Beet sugar, distilled water, lactic acid |
| Composition 76 | Beet sugar, vapor distilled electrolyte enhanced water |
| Composition 77 | Beet sugar, vapor distilled electrolyte enhanced water, sodium |
| | benzoate, citric acid |
| Composition 78 | Beet sugar, vapor distilled electrolyte enhanced water, lactic |
| | acid |
| Composition 79 | Beet sugar, triple filtered water |
| Composition 80 | Beet sugar, triple filtered water, sodium benzoate, citric acid |
| Composition 81 | Beet sugar, triple filtered water, lactic acid |
| Composition 82 | Fruit sugar, water |
| Composition 83 | Fruit sugar, water, sodium benzoate, citric acid |
| Composition 84 | Fruit sugar, water, lactic acid |
| Composition 85 | Fruit sugar, mineral water |
| Composition 86 | Fruit sugar, mineral water, sodium benzoate, citric acid |
| Composition 87 | Fruit sugar, mineral water, lactic acid |
| Composition 88 | Fruit sugar, natural spring water |
| Composition 89 | Fruit sugar, natural spring water, sodium benzoate, citric acid |
| Composition 90 | Fruit sugar, natural spring water, lactic acid |
| Composition 91 | Fruit sugar, filtered water |
| Composition 92 | Fruit sugar, filtered water, sodium benzoate, citric |
| Composition 93 | Fruit sugar, filtered water, lactic acid |
| Composition 94 | Fruit sugar, artesian water |
| Composition 95 | Fruit sugar, artesian water, sodium benzoate, citric acid |
| Composition 96 | Fruit sugar, artesian water, lactic acid |
| Composition 97 | Fruit sugar, purified water |
| Composition 98 | Fruit sugar, purified water, sodium benzoate, citric acid |
| Composition 99 | Fruit sugar, purified water, lactic acid |
| Composition 100 | Fruit sugar, distilled water |
| Composition 101 | Fruit sugar, distilled water, sodium benzoate, citric, acid |
| Composition 102 | Fruit sugar, distilled water, lactic acid |
| Composition 103 | Fruit sugar, vapor distilled electrolyte enhanced water |
| Composition 104 | Fruit sugar, vapor distilled electrolyte enhanced water, sodium |
| | benzoate, citric acid |
| Composition 105 | Fruit sugar, vapor distilled electrolyte enhanced water, lactic |
| | acid |
| Composition 106 | Fruit sugar, triple filtered water |
| Composition 107 | Fruit sugar, triple filtered water, sodium benzoate, citric acid |
| Composition 108 | Fruit sugar, triple filtered water, lactic acid |
| Composition 109 | Brown Sugar, natural spring water |
| Composition 110 | Brown Sugar, natural spring water, sodium benzoate, citric |
| | acid |
| Composition 111 | Brown Sugar, natural spring water, lactic acid |
| Composition 112 | Brown Sugar, filtered water |
| Composition 113 | Brown Sugar, filtered water, sodium benzoate, citric |
| Composition 114 | Brown Sugar, filtered water, lactic acid |
| Composition 115 | Brown Sugar, artesian water |
| Composition 116 | Brown Sugar, artesian water, sodium benzoate, citric acid |
| Composition 117 | Brown Sugar, artesian water, lactic acid |
| Composition 118 | Brown Sugar, purified water |
| Composition 119 | Brown Sugar, purified water, sodium benzoate, citric acid |
| Composition 120 | Brown Sugar, purified water, lactic acid |
| Composition 121 | Brown Sugar, distilled water |
| Composition 122 | Brown Sugar, distilled water, sodium benzoate, citric, acid |
| Composition 123 | Brown Sugar, distilled water, lactic acid |
| Composition 124 | Brown Sugar, vapor distilled electrolyte enhanced water |
| Composition 125 | Brown Sugar, vapor distilled electrolyte enhanced water, |
| | sodium benzoate, citric acid |
| Composition 126 | Brown Sugar, vapor distilled electrolyte enhanced water, lactic |
| | acid |
| Composition 127 | Brown Sugar, triple filtered water |
| Composition 128 | Brown Sugar, triple filtered water, sodium benzoate, citric acid |
| Composition 129 | Brown Sugar, triple filtered water, lactic acid |
| Composition 130 | Honey, natural spring water |
| Composition 131 | Honey, natural spring water, sodium benzoate, citric acid |
| Composition 132 | Honey, natural spring water, lactic acid |
| Composition 133 | Honey, filtered water |
| Composition 134 | Honey, filtered water, sodium benzoate, citric |
| Composition 135 | Honey, filtered water, lactic acid |
| Composition 136 | Honey, artesian water |
| Composition 137 | Honey, artesian water, sodium benzoate, citric acid |
| Composition 138 | Honey, artesian water, lactic acid |
| Composition 139 | Honey, purified water |
| Composition 140 | Honey, purified water, sodium benzoate, citric acid |
| Composition 141 | Honey, purified water, lactic acid |
| Composition 142 | Honey, distilled water |
| Composition 143 | Honey, distilled water, sodium benzoate, citric, acid |
| Composition 144 | Honey, distilled water, lactic acid |
| Composition 145 | Honey, vapor distilled electrolyte enhanced water |
| Composition 146 | Honey, vapor distilled electrolyte enhanced water, sodium |
| | benzoate, citric acid |
| Composition 147 | Honey, vapor distilled electrolyte enhanced water, lactic acid |
| Composition 148 | Honey, triple filtered water |
| Composition 149 | Honey, triple filtered water, sodium benzoate, citric acid |
| Composition 150 | Honey, triple filtered water, lactic acid |
| Composition 151 | Corn syrup, natural spring water |
| Composition 152 | Corn syrup, natural spring water, sodium benzoate, citric acid |
| Composition 153 | Corn syrup, natural spring water, lactic acid |
| Composition 154 | Corn syrup, filtered water |
| Composition 155 | Corn syrup, filtered water, sodium benzoate, citric |
| Composition 156 | Corn syrup, filtered water, lactic acid |
| Composition 157 | Corn syrup, artesian water |
| Composition 158 | Corn syrup, artesian water, sodium benzoate, citric acid |
| Composition 159 | Corn syrup, artesian water, lactic acid |
| Composition 160 | Corn syrup, purified water |
| Composition 161 | Corn syrup, purified water, sodium benzoate, citric acid |
| Composition 162 | Corn syrup, purified water, lactic acid |
| Composition 163 | Corn syrup, distilled water |
| Composition 164 | Corn syrup, distilled water, sodium benzoate, citric, acid |
| Composition 165 | Corn syrup, distilled water, lactic acid |
| Composition 166 | Corn syrup, vapor distilled electrolyte enhanced water |
| Composition 167 | Corn syrup, vapor distilled electrolyte enhanced water, sodium |
| | benzoate, citric acid |
| Composition 168 | Corn syrup, vapor distilled electrolyte enhanced water, lactic |
| | acid |
| Composition 169 | Corn syrup, triple filtered water |
| Composition 170 | Corn syrup, triple filtered water, sodium benzoate, citric acid |
| Composition 171 | Corn syrup, triple filtered water, lactic acid |
| Composition 172 | High fructose corn syrup, natural spring water |
| Composition 173 | High fructose corn syrup, natural spring water, sodium |
| | benzoate, citric acid |
| Composition 174 | High fructose corn syrup, natural spring water, lactic acid |
| Composition 175 | High fructose corn syrup, filtered water |
| Composition 176 | High fructose corn syrup, filtered water, sodium benzoate, |
| | citric |
| Composition 177 | High fructose corn syrup, filtered water, lactic acid |
| Composition 178 | High fructose corn syrup, artesian water |
| Composition 179 | High fructose corn syrup, artesian water, sodium benzoate, |
| | citric acid |
| Composition 180 | High fructose corn syrup, artesian water, lactic acid |
| Composition 181 | High fructose corn syrup, purified water |
| Composition 182 | High fructose corn syrup, purified water, sodium benzoate, |
| | citric acid |
| Composition 183 | High fructose corn syrup, purified water, lactic acid |
| Composition 184 | High fructose corn syrup, distilled water |
| Composition 185 | High fructose corn syrup, distilled water, sodium benzoate, |
| | citric, acid |
| Composition 186 | High fructose corn syrup, distilled water, lactic acid |
| Composition 187 | High fructose corn syrup, vapor distilled electrolyte enhanced |
| | water |
| Composition 188 | High fructose corn syrup, vapor distilled electrolyte enhanced |
| | water, sodium benzoate, citric acid |
| Composition 189 | High fructose corn syrup, vapor distilled electrolyte enhanced |
| | water, lactic acid |
| Composition 190 | High fructose corn syrup, triple filtered water |
| Composition 191 | High fructose corn syrup, triple filtered water, sodium |
| | benzoate, citric acid |
| Composition 192 | High fructose corn syrup, triple filtered water, lactic acid |
| Composition 193 | Turbinado (Demerara) sugar, water |
| Composition 194 | Turbinado (Demerara) sugar, water, sodium benzoate, citric |
| | acid |
| Composition 195 | Turbinado (Demerara) sugar, water, lactic acid |
| Composition 196 | Turbinado (Demerara) sugar, mineral water |
| Composition 197 | Turbinado (Demerara) sugar, mineral water, sodium benzoate, |
| | citric acid |
| Composition 198 | Turbinado (Demerara) sugar, mineral water, lactic acid |
| Composition 199 | Turbinado (Demerara) sugar, natural spring water |
| Composition 200 | Turbinado (Demerara) sugar, natural spring water, sodium |
| | benzoate, citric acid |
| Composition 201 | Turbinado (Demerara) sugar, natural spring water, lactic acid |
| Composition 202 | Turbinado (Demerara) sugar, filtered water |
| Composition 203 | Turbinado (Demerara) sugar, filtered water, sodium benzoate, |
| | citric |
| Composition 204 | Turbinado (Demerara) sugar, filtered water, lactic acid |
| Composition 205 | Turbinado (Demerara) sugar, artesian water |
| Composition 206 | Turbinado (Demerara) sugar, artesian water, sodium benzoate, |
| | citric acid |
| Composition 207 | Turbinado (Demerara) sugar, artesian water, lactic acid |
| Composition 208 | Turbinado (Demerara) sugar, purified water |
| Composition 209 | Turbinado (Demerara) sugar, purified water, sodium |
| | benzoate, citric acid |
| Composition 210 | Turbinado (Demerara) sugar, purified water, lactic acid |
| Composition 211 | Turbinado (Demerara) sugar, distilled water |
| Composition 212 | Turbinado (Demerara) sugar, distilled water, sodium benzoate, |
| | citric, acid |
| Composition 213 | Turbinado (Demerara) sugar, distilled water, lactic acid |
| Composition 214 | Turbinado (Demerara) sugar, vapor distilled electrolyte |
| | enhanced water |
| Composition 215 | Turbinado (Demerara) sugar, vapor distilled electrolyte |
| | enhanced water, sodium benzoate, citric acid |
| Composition 216 | Turbinado (Demerara) sugar, vapor distilled electrolyte |
| | enhanced water, lactic acid |
| Composition 217 | Turbinado (Demerara) sugar, triple filtered water |
| Composition 218 | Turbinado (Demerara) sugar, triple filtered water, sodium |
| | benzoate, citric acid |
| Composition 219 | Turbinado (Demerara) sugar, triple filtered water, lactic acid |

The various ingredients that are used to formulate various compositions of a natural substitute liquid sugar with a brix degree measurement of greater than 67.5 are listed below in Table 2.

TABLE 2
COMPOSITIONS WITH NATURAL SUGAR SUBSTITUTES

| Composition of Liquid | |
|---|---|
| Sugar | Ingredients |
| Composition 1 | Rebiana, erythritol, water |
| Composition 2 | Rebiana, erythritol, water, sodium benzoate, citric acid |
| Composition 3 | Rebiana, erythritol, water, lactic acid |
| Composition 4 | Rebiana, erythritol, mineral water |
| Composition 5 | Rebiana, erythritol, mineral water, sodium benzoate, citric acid |
| Composition 6 | Rebiana, erythritol, mineral water, lactic acid |
| Composition 7 | Rebiana, erythritol, natural spring water |
| Composition 8 | Rebiana, erythritol, natural spring water, sodium benzoate, |
| | citric acid |
| Composition 9 | Rebiana, erythritol, natural spring water, lactic acid |
| Composition 10 | Rebiana, erythritol, filtered water |
| Composition 11 | Rebiana, erythritol, filtered water, sodium benzoate, citric |
| Composition 12 | Rebiana, erythritol, filtered water, lactic acid |
| Composition 13 | Rebiana, erythritol, artesian water |
| Composition 14 | Rebiana, erythritol, artesian water, sodium benzoate, citric acid |
| Composition 15 | Rebiana, erythritol, artesian water, lactic acid |
| Composition 16 | Rebiana, erythritol, purified water |
| Composition 17 | Rebiana, erythritol, purified water, sodium benzoate, citric |
| | acid |
| Composition 18 | Rebiana, erythritol, purified water, lactic acid |
| Composition 19 | Rebiana, erythritol, distilled water |
| Composition 20 | Rebiana, erythritol, distilled water, sodium benzoate, citric, |
| | acid |
| Composition 21 | Rebiana, erythritol, distilled water, lactic acid |
| Composition 22 | Rebiana, erythritol, vapor distilled electrolyte enhanced water |
| Composition 23 | Rebiana, erythritol, vapor distilled electrolyte enhanced water, |
| | sodium benzoate, citric acid |
| Composition 24 | Rebiana, erythritol, vapor distilled electrolyte enhanced water, |
| | lactic acid |
| Composition 25 | Rebiana, erythritol, triple filtered water |
| Composition 26 | Rebiana, erythritol, triple filtered water, sodium benzoate, |
| | citric acid |
| Composition 27 | Rebiana, erythritol, triple filtered water, lactic acid |
| Composition 28 | Organic rebiana, erythritol, water |
| Composition 29 | Organic rebiana, erythritol, water, sodium benzoate, citric acid |
| Composition 30 | Organic rebiana, erythritol, water, lactic acid |
| Composition 31 | Organic rebiana, erythritol, mineral water |
| Composition 32 | Organic rebiana, erythritol, mineral water, sodium benzoate, |
| | citric acid |
| Composition 33 | Organic rebiana, erythritol, mineral water, lactic acid |
| Composition 34 | Organic rebiana, erythritol, natural spring water |
| Composition 35 | Organic rebiana, erythritol, natural spring water, sodium |
| | benzoate, citric acid |
| Composition 36 | Organic rebiana, erythritol, natural spring water, lactic acid |
| Composition 37 | Organic rebiana, erythritol, filtered water |
| Composition 38 | Organic rebiana, erythritol, filtered water, sodium benzoate, |
| | citric |
| Composition 39 | Organic rebiana, erythritol, filtered water, lactic acid |
| Composition 40 | Organic rebiana, erythritol, artesian water |
| Composition 41 | Organic rebiana, erythritol, artesian water, sodium benzoate, |
| | citric acid |
| Composition 42 | Organic rebiana, erythritol, artesian water, lactic acid |
| Composition 43 | Organic rebiana, erythritol, purified water |
| Composition 44 | Organic rebiana, erythritol, purified water, sodium benzoate, |
| | citric acid |
| Composition 45 | Organic rebiana, erythritol, purified water, lactic acid |
| Composition 46 | Organic rebiana, erythritol, distilled water |
| Composition 47 | Organic rebiana, erythritol, distilled water, sodium benzoate, |
| | citric, acid |
| Composition 48 | Organic rebiana, erythritol, distilled water, lactic acid |
| Composition 49 | Organic rebiana, erythritol, vapor distilled electrolyte |
| | enhanced water |
| Composition 50 | Organic rebiana, erythritol, vapor distilled electrolyte |
| | enhanced water, sodium benzoate, citric acid |
| Composition 51 | Organic rebiana, erythritol, vapor distilled electrolyte |
| | enhanced water, lactic acid |
| Composition 52 | Organic rebiana, erythritol, triple filtered water |
| Composition 53 | Organic rebiana, erythritol, triple filtered water, sodium |
| | benzoate, citric acid |
| Composition 54 | Organic rebiana, erythritol, triple filtered water, lactic acid |
| Composition 55 | Stevia, water |
| Composition 56 | Stevia, water, sodium benzoate, citric acid |
| Composition 57 | Stevia, water, lactic acid |
| Composition 58 | Stevia, mineral water |
| Composition 59 | Stevia, mineral water, sodium benzoate, citric acid |
| Composition 60 | Stevia, mineral water, lactic acid |
| Composition 61 | Stevia, natural spring water |
| Composition 62 | Stevia, natural spring water, sodium benzoate, citric acid |
| Composition 63 | Stevia, natural spring water, lactic acid |
| Composition 64 | Stevia, filtered water |
| Composition 65 | Stevia, filtered water, sodium benzoate, citric |
| Composition 66 | Stevia, filtered water, lactic acid |
| Composition 67 | Stevia, artesian water |
| Composition 68 | Stevia, artesian water, sodium benzoate, citric acid |
| Composition 69 | Stevia, artesian water, lactic acid |
| Composition 70 | Stevia, purified water |
| Composition 71 | Stevia, purified water, sodium benzoate, citric acid |
| Composition 72 | Stevia, purified water, lactic acid |
| Composition 73 | Stevia, distilled water |
| Composition 74 | Stevia, distilled water, sodium benzoate, citric, acid |
| Composition 75 | Stevia, distilled water, lactic acid |
| Composition 76 | Stevia, vapor distilled electrolyte enhanced water |
| Composition 77 | Stevia, vapor distilled electrolyte enhanced water, sodium |
| | benzoate, citric acid |
| Composition 78 | Stevia, vapor distilled electrolyte enhanced water, lactic acid |
| Composition 79 | Stevia, triple filtered water |
| Composition 80 | Stevia, triple filtered water, sodium benzoate, citric acid |
| Composition 81 | Stevia, triple filtered water, lactic acid |
| Composition 82 | Agave nectar, water |
| Composition 83 | Agave nectar, water, sodium benzoate, citric acid |
| Composition 84 | Agave nectar, water, lactic acid |
| Composition 85 | Agave nectar, mineral water |
| Composition 86 | Agave nectar, mineral water, sodium benzoate, citric acid |
| Composition 87 | Agave nectar, mineral water, lactic acid |
| Composition 88 | Agave nectar, natural spring water |
| Composition 89 | Agave nectar, natural spring water, sodium benzoate, citric |
| | acid |
| Composition 90 | Agave nectar, natural spring water, lactic acid |
| Composition 91 | Agave nectar, filtered water |
| Composition 92 | Agave nectar, filtered water, sodium benzoate, citric |
| Composition 93 | Agave nectar, filtered water, lactic acid |
| Composition 94 | Agave nectar, artesian water |
| Composition 95 | Agave nectar, artesian water, sodium benzoate, citric acid |
| Composition 96 | Agave nectar, artesian water, lactic acid |
| Composition 97 | Agave nectar, purified water |
| Composition 98 | Agave nectar, purified water, sodium benzoate, citric acid |
| Composition 99 | Agave nectar, purified water, lactic acid |
| Composition 100 | Agave nectar, distilled water |
| Composition 101 | Agave nectar, distilled water, sodium benzoate, citric, acid |
| Composition 102 | Agave nectar, distilled water, lactic acid |
| Composition 103 | Agave nectar, vapor distilled electrolyte enhanced water |
| Composition 104 | Agave nectar, vapor distilled electrolyte enhanced water, |
| | sodium benzoate, citric acid |
| Composition 105 | Agave nectar, vapor distilled electrolyte enhanced water, lactic |
| | acid |
| Composition 106 | Agave nectar, triple filtered water |
| Composition 107 | Agave nectar, triple filtered water, sodium benzoate, citric acid |
| Composition 108 | Agave nectar, triple filtered water, lactic acid |
| Composition 109 | Brazzein, water |
| Composition 110 | Brazzein, water, sodium benzoate, citric acid |
| Composition 111 | Brazzein, water, lactic acid |
| Composition 112 | Brazzein, mineral water |
| Composition 113 | Brazzein, mineral water, sodium benzoate, citric acid |
| Composition 114 | Brazzein, mineral water, lactic acid |
| Composition 115 | Brazzein, natural spring water |
| Composition 116 | Brazzein, natural spring water, sodium benzoate, citric acid |
| Composition 117 | Brazzein, natural spring water, lactic acid |
| Composition 118 | Brazzein, filtered water |
| Composition 119 | Brazzein, filtered water, sodium benzoate, citric |
| Composition 120 | Brazzein, filtered water, lactic acid |
| Composition 121 | Brazzein, artesian water |
| Composition 122 | Brazzein, artesian water, sodium benzoate, citric acid |
| Composition 123 | Brazzein, artesian water, lactic acid |
| Composition 124 | Brazzein, purified water |
| Composition 125 | Brazzein, purified water, sodium benzoate, citric acid |
| Composition 126 | Brazzein, purified water, lactic acid |
| Composition 127 | Brazzein, distilled water |
| Composition 128 | Brazzein, distilled water, sodium benzoate, citric, acid |
| Composition 129 | Brazzein, distilled water, lactic acid |
| Composition 130 | Brazzein, vapor distilled electrolyte enhanced water |
| Composition 131 | Brazzein, vapor distilled electrolyte enhanced water, sodium |
| | benzoate, citric acid |
| Composition 132 | Brazzein, vapor distilled electrolyte enhanced water, lactic acid |
| Composition 133 | Brazzein, triple filtered water |
| Composition 134 | Brazzein, triple filtered water, sodium benzoate, citric acid |
| Composition 135 | Brazzein, triple filtered water, lactic acid |
| Composition 136 | Curculin, water |
| Composition 137 | Curculin, water, sodium benzoate, citric acid |
| Composition 138 | Curculin, water, lactic acid |
| Composition 139 | Curculin, mineral water |
| Composition 140 | Curculin, mineral water, sodium benzoate, citric acid |
| Composition 141 | Curculin, mineral water, lactic acid |
| Composition 142 | Curculin, natural spring water |
| Composition 143 | Curculin, natural spring water, sodium benzoate, citric acid |
| Composition 144 | Curculin, natural spring water, lactic acid |
| Composition 145 | Curculin, filtered water |
| Composition 146 | Curculin, filtered water, sodium benzoate, citric |
| Composition 147 | Curculin, filtered water, lactic acid |
| Composition 148 | Curculin, artesian water |
| Composition 149 | Curculin, artesian water, sodium benzoate, citric acid |
| Composition 150 | Curculin, artesian water, lactic acid |
| Composition 151 | Curculin, purified water |
| Composition 152 | Curculin, purified water, sodium benzoate, citric acid |
| Composition 153 | Curculin, purified water, lactic acid |
| Composition 154 | Curculin, distilled water |
| Composition 155 | Curculin, distilled water, sodium benzoate, citric, acid |
| Composition 156 | Curculin, distilled water, lactic acid |
| Composition 157 | Curculin, vapor distilled electrolyte enhanced water |
| Composition 158 | Curculin, vapor distilled electrolyte enhanced water, sodium |
| | benzoate, citric acid |
| Composition 159 | Curculin, vapor distilled electrolyte enhanced water, lactic acid |
| Composition 160 | Curculin, triple filtered water |
| Composition 161 | Curculin, triple filtered water, sodium benzoate, citric acid |
| Composition 162 | Curculin, triple filtered water, lactic acid |
| Composition 163 | Erythritol, water |
| Composition 164 | Erythritol, water, sodium benzoate, citric acid |
| Composition 165 | Erythritol, water, lactic acid |
| Composition 166 | Erythritol, mineral water |
| Composition 167 | Erythritol, mineral water, sodium benzoate, citric acid |
| Composition 168 | Erythritol, mineral water, lactic acid |
| Composition 169 | Erythritol, natural spring water |
| Composition 170 | Erythritol, natural spring water, sodium benzoate, citric acid |
| Composition 171 | Erythritol, natural spring water, lactic acid |
| Composition 172 | Erythritol, filtered water |
| Composition 173 | Erythritol, filtered water, sodium benzoate, citric |
| Composition 174 | Erythritol, filtered water, lactic acid |
| Composition 175 | Erythritol, artesian water |
| Composition 176 | Erythritol, artesian water, sodium benzoate, citric acid |
| Composition 177 | Erythritol, artesian water, lactic acid |
| Composition 178 | Erythritol, purified water |
| Composition 179 | Erythritol, purified water, sodium benzoate, citric acid |
| Composition 180 | Erythritol, purified water, lactic acid |
| Composition 181 | Erythritol, distilled water |
| Composition 182 | Erythritol, distilled water, sodium benzoate, citric, acid |
| Composition 183 | Erythritol, distilled water, lactic acid |
| Composition 184 | Erythritol, vapor distilled electrolyte enhanced water |
| Composition 185 | Erythritol, vapor distilled electrolyte enhanced water, sodium |
| | benzoate, citric acid |
| Composition 186 | Erythritol, vapor distilled electrolyte enhanced water, lactic |
| | acid |
| Composition 187 | Erythritol, triple filtered water |
| Composition 188 | Erythritol, triple filtered water, sodium benzoate, citric acid |
| Composition 189 | Erythritol, triple filtered water, lactic acid |
| Composition 190 | Fructose, water |
| Composition 191 | Fructose, water, sodium benzoate, citric acid |
| Composition 192 | Fructose, water, lactic acid |
| Composition 193 | Fructose, mineral water |
| Composition 194 | Fructose, mineral water, sodium benzoate, citric acid |
| Composition 195 | Fructose, mineral water, lactic acid |
| Composition 196 | Fructose, natural spring water |
| Composition 197 | Fructose, natural spring water, sodium benzoate, citric acid |
| Composition 198 | Fructose, natural spring water, lactic acid |
| Composition 199 | Fructose, filtered water |
| Composition 200 | Fructose, filtered water, sodium benzoate, citric |
| Composition 201 | Fructose, filtered water, lactic acid |
| Composition 202 | Fructose, artesian water |
| Composition 203 | Fructose, artesian water, sodium benzoate, citric acid |
| Composition 204 | Fructose, artesian water, lactic acid |
| Composition 205 | Fructose, purified water |
| Composition 206 | Fructose, purified water, sodium benzoate, citric acid |
| Composition 207 | Fructose, purified water, lactic acid |
| Composition 208 | Fructose, distilled water |
| Composition 209 | Fructose, distilled water, sodium benzoate, citric, acid |
| Composition 210 | Fructose, distilled water, lactic acid |
| Composition 211 | Fructose, vapor distilled electrolyte enhanced water |
| Composition 212 | Fructose, vapor distilled electrolyte enhanced water, sodium |
| | benzoate, citric acid |
| Composition 213 | Fructose, vapor distilled electrolyte enhanced water, lactic acid |
| Composition 214 | Fructose, triple filtered water |
| Composition 215 | Fructose, triple filtered water, sodium benzoate, citric acid |
| Composition 216 | Fructose, triple filtered water, lactic acid |
| Composition 217 | Glycyrrhizin, water |
| Composition 218 | Glycyrrhizin, water, sodium benzoate, citric acid |
| Composition 219 | Glycyrrhizin, water, lactic acid |
| Composition 220 | Glycyrrhizin, mineral water |
| Composition 221 | Glycyrrhizin, mineral water, sodium benzoate, citric acid |
| Composition 222 | Glycyrrhizin, mineral water, lactic acid |
| Composition 223 | Glycyrrhizin, natural spring water |
| Composition 224 | Glycyrrhizin, natural spring water, sodium benzoate, citric |
| | acid |
| Composition 225 | Glycyrrhizin, natural spring water, lactic acid |
| Composition 226 | Glycyrrhizin, filtered water |
| Composition 227 | Glycyrrhizin, filtered water, sodium benzoate, citric |
| Composition 228 | Glycyrrhizin, filtered water, lactic acid |
| Composition 229 | Glycyrrhizin, artesian water |
| Composition 230 | Glycyrrhizin, artesian water, sodium benzoate, citric acid |
| Composition 231 | Glycyrrhizin, artesian water, lactic acid |
| Composition 232 | Glycyrrhizin, purified water |
| Composition 233 | Glycyrrhizin, purified water, sodium benzoate, citric acid |
| Composition 234 | Glycyrrhizin, purified water, lactic acid |
| Composition 235 | Glycyrrhizin, distilled water |
| Composition 236 | Glycyrrhizin, distilled water, sodium benzoate, citric, acid |
| Composition 237 | Glycyrrhizin, distilled water, lactic acid |
| Composition 238 | Glycyrrhizin, vapor distilled electrolyte enhanced water |
| Composition 239 | Glycyrrhizin, vapor distilled electrolyte enhanced water, |
| | sodium benzoate, citric acid |
| Composition 240 | Glycyrrhizin, vapor distilled electrolyte enhanced water, lactic |
| | acid |
| Composition 241 | Glycyrrhizin, triple filtered water |
| Composition 242 | Glycyrrhizin, triple filtered water, sodium benzoate, citric acid |
| Composition 243 | Glycyrrhizin, triple filtered water, lactic acid |
| Composition 244 | Glycerol, water |
| Composition 245 | Glycerol, water, sodium benzoate, citric acid |
| Composition 246 | Glycerol, water, lactic acid |
| Composition 247 | Glycerol, mineral water |
| Composition 248 | Glycerol, mineral water, sodium benzoate, citric acid |
| Composition 249 | Glycerol, mineral water, lactic acid |
| Composition 250 | Glycerol, natural spring water |
| Composition 251 | Glycerol, natural spring water, sodium benzoate, citric acid |
| Composition 252 | Glycerol, natural spring water, lactic acid |
| Composition 253 | Glycerol, filtered water |
| Composition 254 | Glycerol, filtered water, sodium benzoate, citric |
| Composition 255 | Glycerol, filtered water, lactic acid |
| Composition 256 | Glycerol, artesian water |
| Composition 257 | Glycerol, artesian water, sodium benzoate, citric acid |
| Composition 258 | Glycerol, artesian water, lactic acid |
| Composition 259 | Glycerol, purified water |
| Composition 260 | Glycerol, purified water, sodium benzoate, citric acid |
| Composition 261 | Glycerol, purified water, lactic acid |
| Composition 262 | Glycerol, distilled water |
| Composition 263 | Glycerol, distilled water, sodium benzoate, citric, acid |
| Composition 264 | Glycerol, distilled water, lactic acid |
| Composition 265 | Glycerol, vapor distilled electrolyte enhanced water |
| Composition 266 | Glycerol, vapor distilled electrolyte enhanced water, sodium |
| | benzoate, citric acid |
| Composition 267 | Glycerol, vapor distilled electrolyte enhanced water, lactic acid |
| Composition 268 | Glycerol, triple filtered water |
| Composition 269 | Glycerol, triple filtered water, sodium benzoate, citric acid |
| Composition 270 | Glycerol, triple filtered water, lactic acid |
| Composition 271 | Hydrogenated starch hydrolysates, water |
| Composition 272 | Hydrogenated starch hydrolysates, water, sodium benzoate, |
| | citric acid |
| Composition 273 | Hydrogenated starch hydrolysates, water, lactic acid |
| Composition 274 | Hydrogenated starch hydrolysates, mineral water |
| Composition 275 | Hydrogenated starch hydrolysates, mineral water, sodium |
| | benzoate, citric acid |
| Composition 276 | Hydrogenated starch hydrolysates, mineral water, lactic acid |
| Composition 277 | Hydrogenated starch hydrolysates, natural spring water |
| Composition 278 | Hydrogenated starch hydrolysates, natural spring water, |
| | sodium benzoate, citric acid |
| Composition 279 | Hydrogenated starch hydrolysates, natural spring water, lactic |
| | acid |
| Composition 280 | Hydrogenated starch hydrolysates, filtered water |
| Composition 281 | Hydrogenated starch hydrolysates, filtered water, sodium |
| | benzoate, citric |
| Composition 282 | Hydrogenated starch hydrolysates, filtered water, lactic acid |
| Composition 283 | Hydrogenated starch hydrolysates, artesian water |
| Composition 284 | Hydrogenated starch hydrolysates, artesian water, sodium |
| | benzoate, citric acid |
| Composition 285 | Hydrogenated starch hydrolysates, artesian water, lactic acid |
| Composition 286 | Hydrogenated starch hydrolysates, purified water |
| Composition 287 | Hydrogenated starch hydrolysates, purified water, sodium |
| | benzoate, citric acid |
| Composition 288 | Hydrogenated starch hydrolysates, purified water, lactic acid |
| Composition 289 | Hydrogenated starch hydrolysates, distilled water |
| Composition 290 | Hydrogenated starch hydrolysates, distilled water, sodium |
| | benzoate, citric, acid |
| Composition 291 | Hydrogenated starch hydrolysates, distilled water, lactic acid |
| Composition 292 | Hydrogenated starch hydrolysates, vapor distilled electrolyte |
| | enhanced water |
| Composition 293 | Hydrogenated starch hydrolysates, vapor distilled electrolyte |
| | enhanced water, sodium benzoate, citric acid |
| Composition 294 | Hydrogenated starch hydrolysates, vapor distilled electrolyte |
| | enhanced water, lactic acid |
| Composition 295 | Hydrogenated starch hydrolysates, triple filtered water |
| Composition 296 | Hydrogenated starch hydrolysates, triple filtered water, |
| | sodium benzoate, citric acid |
| Composition 297 | Hydrogenated starch hydrolysates, triple filtered water, lactic |
| | acid |
| Composition 298 | Inulin, water |
| Composition 299 | Inulin, water, sodium benzoate, citric acid |
| Composition 300 | Inulin, water, lactic acid |
| Composition 301 | Inulin, mineral water |
| Composition 302 | Inulin, mineral water, sodium benzoate, citric acid |
| Composition 303 | Inulin, mineral water, lactic acid |
| Composition 304 | Inulin, natural spring water |
| Composition 305 | Inulin, natural spring water, sodium benzoate, citric acid |
| Composition 306 | Inulin, natural spring water, lactic acid |
| Composition 307 | Inulin, filtered water |
| Composition 308 | Inulin, filtered water, sodium benzoate, citric |
| Composition 309 | Inulin, filtered water, lactic acid |
| Composition 310 | Inulin, artesian water |
| Composition 311 | Inulin, artesian water, sodium benzoate, citric acid |
| Composition 312 | Inulin, artesian water, lactic acid |
| Composition 313 | Inulin, purified water |
| Composition 314 | Inulin, purified water, sodium benzoate, citric acid |
| Composition 315 | Inulin, purified water, lactic acid |
| Composition 316 | Inulin, distilled water |
| Composition 317 | Inulin, distilled water, sodium benzoate, citric, acid |
| Composition 318 | Inulin, distilled water, lactic acid |
| Composition 319 | Inulin, vapor distilled electrolyte enhanced water |
| Composition 320 | Inulin, vapor distilled electrolyte enhanced water, sodium |
| | benzoate, citric acid |
| Composition 321 | Inulin, vapor distilled electrolyte enhanced water, lactic acid |
| Composition 322 | Inulin, triple filtered water |
| Composition 323 | Inulin, triple filtered water, sodium benzoate, citric acid |
| Composition 324 | Inulin, triple filtered water, lactic acid |
| Composition 325 | Isomalt, water |
| Composition 326 | Isomalt, water, sodium benzoate, citric acid |
| Composition 327 | Isomalt, water, lactic acid |
| Composition 328 | Isomalt, mineral water |
| Composition 329 | Isomalt, mineral water, sodium benzoate, citric acid |
| Composition 330 | Isomalt, mineral water, lactic acid |
| Composition 331 | Isomalt, natural spring water |
| Composition 332 | Isomalt, natural spring water, sodium benzoate, citric acid |
| Composition 333 | Isomalt, natural spring water, lactic acid |
| Composition 334 | Isomalt, filtered water |
| Composition 335 | Isomalt, filtered water, sodium benzoate, citric |
| Composition 336 | Isomalt, filtered water, lactic acid |
| Composition 337 | Isomalt, artesian water |
| Composition 338 | Isomalt, artesian water, sodium benzoate, citric acid |
| Composition 339 | Isomalt, artesian water, lactic acid |
| Composition 340 | Isomalt, purified water |
| Composition 341 | Isomalt, purified water, sodium benzoate, citric acid |
| Composition 342 | Isomalt, purified water, lactic acid |
| Composition 343 | Isomalt, distilled water |
| Composition 344 | Isomalt, distilled water, sodium benzoate, citric, acid |
| Composition 345 | Isomalt, distilled water, lactic acid |
| Composition 346 | Isomalt, vapor distilled electrolyte enhanced water |
| Composition 347 | Isomalt, vapor distilled electrolyte enhanced water, sodium |
| | benzoate, citric acid |
| Composition 348 | Isomalt, vapor distilled electrolyte enhanced water, lactic acid |
| Composition 349 | Isomalt, triple filtered water |
| Composition 350 | Isomalt, triple filtered water, sodium benzoate, citric acid |
| Composition 351 | Isomalt, triple filtered water, lactic acid |
| Composition 352 | Lactitol, water |
| Composition 353 | Lactitol, water, sodium benzoate, citric acid |
| Composition 354 | Lactitol, water, lactic acid |
| Composition 355 | Lactitol, mineral water |
| Composition 356 | Lactitol, mineral water, sodium benzoate, citric acid |
| Composition 357 | Lactitol, mineral water, lactic acid |
| Composition 358 | Lactitol, natural spring water |
| Composition 359 | Lactitol, natural spring water, sodium benzoate, citric acid |
| Composition 360 | Lactitol, natural spring water, lactic acid |
| Composition 361 | Lactitol, filtered water |
| Composition 362 | Lactitol, filtered water, sodium benzoate, citric |
| Composition 363 | Lactitol, filtered water, lactic acid |
| Composition 364 | Lactitol, artesian water |
| Composition 365 | Lactitol, artesian water, sodium benzoate, citric acid |
| Composition 366 | Lactitol, artesian water, lactic acid |
| Composition 367 | Lactitol, purified water |
| Composition 368 | Lactitol, purified water, sodium benzoate, citric acid |
| Composition 369 | Lactitol, purified water, lactic acid |
| Composition 370 | Lactitol, distilled water |
| Composition 371 | Lactitol, distilled water, sodium benzoate, citric, acid |
| Composition 372 | Lactitol, distilled water, lactic acid |
| Composition 373 | Lactitol, vapor distilled electrolyte enhanced water |
| Composition 374 | Lactitol, vapor distilled electrolyte enhanced water, sodium |
| | benzoate, citric acid |
| Composition 375 | Lactitol, vapor distilled electrolyte enhanced water, lactic acid |
| Composition 376 | Lactitol, triple filtered water |
| Composition 377 | Lactitol, triple filtered water, sodium benzoate, citric acid |
| Composition 378 | Lactitol, triple filtered water, lactic acid |
| Composition 379 | Lo Han Guo, water |
| Composition 380 | Lo Han Guo, water, sodium benzoate, citric acid |
| Composition 381 | Lo Han Guo, water, lactic acid |
| Composition 382 | Lo Han Guo, mineral water |
| Composition 383 | Lo Han Guo, mineral water, sodium benzoate, citric acid |
| Composition 384 | Lo Han Guo, mineral water, lactic acid |
| Composition 385 | Lo Han Guo, natural spring water |
| Composition 386 | Lo Han Guo, natural spring water, sodium benzoate, citric |
| | acid |
| Composition 387 | Lo Han Guo, natural spring water, lactic acid |
| Composition 388 | Lo Han Guo, filtered water |
| Composition 389 | Lo Han Guo, filtered water, sodium benzoate, citric |
| Composition 390 | Lo Han Guo, filtered water, lactic acid |
| Composition 391 | Lo Han Guo, artesian water |
| Composition 392 | Lo Han Guo, artesian water, sodium benzoate, citric acid |
| Composition 393 | Lo Han Guo, artesian water, lactic acid |
| Composition 394 | Lo Han Guo, purified water |
| Composition 395 | Lo Han Guo, purified water, sodium benzoate, citric acid |
| Composition 396 | Lo Han Guo, purified water, lactic acid |
| Composition 397 | Lo Han Guo, distilled water |
| Composition 398 | Lo Han Guo, distilled water, sodium benzoate, citric, acid |
| Composition 399 | Lo Han Guo, distilled water, lactic acid |
| Composition 400 | Lo Han Guo, vapor distilled electrolyte enhanced water |
| Composition 401 | Lo Han Guo, vapor distilled electrolyte enhanced water, |
| | sodium benzoate, citric acid |
| Composition 402 | Lo Han Guo, vapor distilled electrolyte enhanced water, lactic |
| | acid |
| Composition 403 | Lo Han Guo, triple filtered water |
| Composition 404 | Lo Han Guo, triple filtered water, sodium benzoate, citric acid |
| Composition 405 | Lo Han Guo, triple filtered water, lactic acid |
| Composition 406 | Mabinlin, water |
| Composition 407 | Mabinlin, water, sodium benzoate, citric acid |
| Composition 408 | Mabinlin, water, lactic acid |
| Composition 409 | Mabinlin, mineral water |
| Composition 410 | Mabinlin, mineral water, sodium benzoate, citric acid |
| Composition 411 | Mabinlin, mineral water, lactic acid |
| Composition 412 | Mabinlin, natural spring water |
| Composition 413 | Mabinlin, natural spring water, sodium benzoate, citric acid |
| Composition 414 | Mabinlin, natural spring water, lactic acid |
| Composition 415 | Mabinlin, filtered water |
| Composition 416 | Mabinlin, filtered water, sodium benzoate, citric |
| Composition 417 | Mabinlin, filtered water, lactic acid |
| Composition 418 | Mabinlin, artesian water |
| Composition 419 | Mabinlin, artesian water, sodium benzoate, citric acid |
| Composition 420 | Mabinlin, artesian water, lactic acid |
| Composition 421 | Mabinlin, purified water |
| Composition 422 | Mabinlin, purified water, sodium benzoate, citric acid |
| Composition 423 | Mabinlin, purified water, lactic acid |
| Composition 424 | Mabinlin, distilled water |
| Composition 425 | Mabinlin, distilled water, sodium benzoate, citric, acid |
| Composition 426 | Mabinlin, distilled water, lactic acid |
| Composition 427 | Mabinlin, vapor distilled electrolyte enhanced water |
| Composition 428 | Mabinlin, vapor distilled electrolyte enhanced water, sodium |
| | benzoate, citric acid |
| Composition 429 | Mabinlin, vapor distilled electrolyte enhanced water, lactic acid |
| Composition 430 | Mabinlin, triple filtered water |
| Composition 431 | Mabinlin, triple filtered water, sodium benzoate, citric acid |
| Composition 432 | Mabinlin, triple filtered water, lactic acid |
| Composition 433 | Maltitol, water |
| Composition 434 | Maltitol, water, sodium benzoate, citric acid |
| Composition 435 | Maltitol, water, lactic acid |
| Composition 436 | Maltitol, mineral water |
| Composition 437 | Maltitol, mineral water, sodium benzoate, citric acid |
| Composition 438 | Maltitol, mineral water, lactic acid |
| Composition 439 | Maltitol, natural spring water |
| Composition 440 | Maltitol, natural spring water, sodium benzoate, citric acid |
| Composition 441 | Maltitol, natural spring water, lactic acid |
| Composition 442 | Maltitol, filtered water |
| Composition 443 | Maltitol, filtered water, sodium benzoate, citric |
| Composition 444 | Maltitol, filtered water, lactic acid |
| Composition 445 | Maltitol, artesian water |
| Composition 446 | Maltitol, artesian water, sodium benzoate, citric acid |
| Composition 447 | Maltitol, artesian water, lactic acid |
| Composition 448 | Maltitol, purified water |
| Composition 449 | Maltitol, purified water, sodium benzoate, citric acid |
| Composition 450 | Maltitol, purified water, lactic acid |
| Composition 451 | Maltitol, distilled water |
| Composition 452 | Maltitol, distilled water, sodium benzoate, citric, acid |
| Composition 453 | Maltitol, distilled water, lactic acid |
| Composition 454 | Maltitol, vapor distilled electrolyte enhanced water |
| Composition 455 | Maltitol, vapor distilled electrolyte enhanced water, sodium |
| | benzoate, citric acid |
| Composition 456 | Maltitol, vapor distilled electrolyte enhanced water, lactic acid |
| Composition 457 | Maltitol, triple filtered water |
| Composition 458 | Maltitol, triple filtered water, sodium benzoate, citric acid |
| Composition 459 | Maltitol, triple filtered water, lactic acid |
| Composition 460 | Maltooligosaccharide, water |
| Composition 461 | Maltooligosaccharide, water, sodium benzoate, citric acid |
| Composition 462 | Maltooligosaccharide, water, lactic acid |
| Composition 463 | Maltooligosaccharide, mineral water |
| Composition 464 | Maltooligosaccharide, mineral water, sodium benzoate, citric |
| | acid |
| Composition 465 | Maltooligosaccharide, mineral water, lactic acid |
| Composition 466 | Maltooligosaccharide, natural spring water |
| Composition 467 | Maltooligosaccharide, natural spring water, sodium benzoate, |
| | citric acid |
| Composition 468 | Maltooligosaccharide, natural spring water, lactic acid |
| Composition 469 | Maltooligosaccharide, filtered water |
| Composition 470 | Maltooligosaccharide, filtered water, sodium benzoate, citric |
| Composition 471 | Maltooligosaccharide, filtered water, lactic acid |
| Composition 472 | Maltooligosaccharide, artesian water |
| Composition 473 | Maltooligosaccharide, artesian water, sodium benzoate, citric |
| | acid |
| Composition 474 | Maltooligosaccharide, artesian water, lactic acid |
| Composition 475 | Maltooligosaccharide, purified water |
| Composition 476 | Maltooligosaccharide, purified water, sodium benzoate, citric |
| | acid |
| Composition 477 | Maltooligosaccharide, purified water, lactic acid |
| Composition 478 | Maltooligosaccharide, distilled water |
| Composition 479 | Maltooligosaccharide, distilled water, sodium benzoate, citric, |
| | acid |
| Composition 480 | Maltooligosaccharide, distilled water, lactic acid |
| Composition 481 | Maltooligosaccharide, vapor distilled electrolyte enhanced |
| | water |
| Composition 482 | Maltooligosaccharide, vapor distilled electrolyte enhanced |
| | water, sodium benzoate, citric acid |
| Composition 483 | Maltooligosaccharide, vapor distilled electrolyte enhanced |
| | water, lactic acid |
| Composition 484 | Maltooligosaccharide, triple filtered water |
| Composition 485 | Maltooligosaccharide, triple filtered water, sodium benzoate, |
| | citric acid |
| Composition 486 | Maltooligosaccharide, triple filtered water, lactic acid |
| Composition 487 | Mannitol, water |
| Composition 488 | Mannitol, water, sodium benzoate, citric acid |
| Composition 489 | Mannitol, water, lactic acid |
| Composition 490 | Mannitol, mineral water |
| Composition 491 | Mannitol, mineral water, sodium benzoate, citric acid |
| Composition 492 | Mannitol, mineral water, lactic acid |
| Composition 493 | Mannitol, natural spring water |
| Composition 494 | Mannitol, natural spring water, sodium benzoate, citric acid |
| Composition 495 | Mannitol, natural spring water, lactic acid |
| Composition 496 | Mannitol, filtered water |
| Composition 497 | Mannitol, filtered water, sodium benzoate, citric |
| Composition 498 | Mannitol, filtered water, lactic acid |
| Composition 499 | Mannitol, artesian water |
| Composition 500 | Mannitol, artesian water, sodium benzoate, citric acid |
| Composition 501 | Mannitol, artesian water, lactic acid |
| Composition 502 | Mannitol, purified water |
| Composition 503 | Mannitol, purified water, sodium benzoate, citric acid |
| Composition 504 | Mannitol, purified water, lactic acid |
| Composition 505 | Mannitol, distilled water |
| Composition 506 | Mannitol, distilled water, sodium benzoate, citric, acid |
| Composition 507 | Mannitol, distilled water, lactic acid |
| Composition 508 | Mannitol, vapor distilled electrolyte enhanced water |
| Composition 509 | Mannitol, vapor distilled electrolyte enhanced water, sodium |
| | benzoate, citric acid |
| Composition 510 | Mannitol, vapor distilled electrolyte enhanced water, lactic |
| | acid |
| Composition 511 | Mannitol, triple filtered water |
| Composition 512 | Mannitol, triple filtered water, sodium benzoate, citric acid |
| Composition 513 | Mannitol, triple filtered water, lactic acid |
| Composition 514 | Miraculin, water |
| Composition 515 | Miraculin, water, sodium benzoate, citric acid |
| Composition 516 | Miraculin, water, lactic acid |
| Composition 517 | Miraculin, mineral water |
| Composition 518 | Miraculin, mineral water, sodium benzoate, citric acid |
| Composition 519 | Miraculin, mineral water, lactic acid |
| Composition 520 | Miraculin, natural spring water |
| Composition 521 | Miraculin, natural spring water, sodium benzoate, citric acid |
| Composition 522 | Miraculin, natural spring water, lactic acid |
| Composition 523 | Miraculin, filtered water |
| Composition 524 | Miraculin, filtered water, sodium benzoate, citric |
| Composition 525 | Miraculin, filtered water, lactic acid |
| Composition 526 | Miraculin, artesian water |
| Composition 527 | Miraculin, artesian water, sodium benzoate, citric acid |
| Composition 528 | Miraculin, artesian water, lactic acid |
| Composition 529 | Miraculin, purified water |
| Composition 530 | Miraculin, purified water, sodium benzoate, citric acid |
| Composition 531 | Miraculin, purified water, lactic acid |
| Composition 532 | Miraculin, distilled water |
| Composition 533 | Miraculin, distilled water, sodium benzoate, citric, acid |
| Composition 534 | Miraculin, distilled water, lactic acid |
| Composition 535 | Miraculin, vapor distilled electrolyte enhanced water |
| Composition 536 | Miraculin, vapor distilled electrolyte enhanced water, sodium |
| | benzoate, citric acid |
| Composition 537 | Miraculin, vapor distilled electrolyte enhanced water, lactic |
| | acid |
| Composition 538 | Miraculin, triple filtered water |
| Composition 539 | Miraculin, triple filtered water, sodium benzoate, citric acid |
| Composition 540 | Miraculin, triple filtered water, lactic acid |
| Composition 541 | Monatin, water |
| Composition 542 | Monatin, water, sodium benzoate, citric acid |
| Composition 543 | Monatin, water, lactic acid |
| Composition 544 | Monatin, mineral water |
| Composition 545 | Monatin, mineral water, sodium benzoate, citric acid |
| Composition 546 | Monatin, mineral water, lactic acid |
| Composition 547 | Monatin, natural spring water |
| Composition 548 | Monatin, natural spring water, sodium benzoate, citric acid |
| Composition 549 | Monatin, natural spring water, lactic acid |
| Composition 550 | Monatin, filtered water |
| Composition 551 | Monatin, filtered water, sodium benzoate, citric |
| Composition 552 | Monatin, filtered water, lactic acid |
| Composition 553 | Monatin, artesian water |
| Composition 554 | Monatin, artesian water, sodium benzoate, citric acid |
| Composition 555 | Monatin, artesian water, lactic acid |
| Composition 556 | Monatin, purified water |
| Composition 557 | Monatin, purified water, sodium benzoate, citric acid |
| Composition 558 | Monatin, purified water, lactic acid |
| Composition 559 | Monatin, distilled water |
| Composition 560 | Monatin, distilled water, sodium benzoate, citric, acid |
| Composition 561 | Monatin, distilled water, lactic acid |
| Composition 562 | Monatin, vapor distilled electrolyte enhanced water |
| Composition 563 | Monatin, vapor distilled electrolyte enhanced water, sodium |
| | benzoate, citric acid |
| Composition 564 | Monatin, vapor distilled electrolyte enhanced water, lactic acid |
| Composition 565 | Monatin, triple filtered water |
| Composition 566 | Monatin, triple filtered water, sodium benzoate, citric acid |
| Composition 567 | Monatin, triple filtered water, lactic acid |
| Composition 568 | Monellin, water |
| Composition 569 | Monellin, water, sodium benzoate, citric acid |
| Composition 570 | Monellin, water, lactic acid |
| Composition 571 | Monellin, mineral water |
| Composition 572 | Monellin, mineral water, sodium benzoate, citric acid |
| Composition 573 | Monellin, mineral water, lactic acid |
| Composition 574 | Monellin, natural spring water |
| Composition 575 | Monellin, natural spring water, sodium benzoate, citric acid |
| Composition 576 | Monellin, natural spring water, lactic acid |
| Composition 577 | Monellin, filtered water |
| Composition 578 | Monellin, filtered water, sodium benzoate, citric |
| Composition 579 | Monellin, filtered water, lactic acid |
| Composition 580 | Monellin, artesian water |
| Composition 581 | Monellin, artesian water, sodium benzoate, citric acid |
| Composition 582 | Monellin, artesian water, lactic acid |
| Composition 583 | Monellin, purified water |
| Composition 584 | Monellin, purified water, sodium benzoate, citric acid |
| Composition 585 | Monellin, purified water, lactic acid |
| Composition 586 | Monellin, distilled water |
| Composition 587 | Monellin, distilled water, sodium benzoate, citric, acid |
| Composition 588 | Monellin, distilled water, lactic acid |
| Composition 589 | Monellin, vapor distilled electrolyte enhanced water |
| Composition 590 | Monellin, vapor distilled electrolyte enhanced water, sodium |
| | benzoate, citric acid |
| Composition 591 | Monellin, vapor distilled electrolyte enhanced water, lactic acid |
| Composition 592 | Monellin, triple filtered water |
| Composition 593 | Monellin, triple filtered water, sodium benzoate, citric acid |
| Composition 594 | Monellin, triple filtered water, lactic acid |
| Composition 595 | Pentadin, water |
| Composition 596 | Pentadin, water, sodium benzoate, citric acid |
| Composition 597 | Pentadin, water, lactic acid |
| Composition 598 | Pentadin, mineral water |
| Composition 599 | Pentadin, mineral water, sodium benzoate, citric acid |
| Composition 600 | Pentadin, mineral water, lactic acid |
| Composition 601 | Pentadin, natural spring water |
| Composition 602 | Pentadin, natural spring water, sodium benzoate, citric acid |
| Composition 603 | Pentadin, natural spring water, lactic acid |
| Composition 604 | Pentadin, filtered water |
| Composition 605 | Pentadin, filtered water, sodium benzoate, citric |
| Composition 606 | Pentadin, filtered water, lactic acid |
| Composition 607 | Pentadin, artesian water |
| Composition 608 | Pentadin, artesian water, sodium benzoate, citric acid |
| Composition 609 | Pentadin, artesian water, lactic acid |
| Composition 610 | Pentadin, purified water |
| Composition 611 | Pentadin, purified water, sodium benzoate, citric acid |
| Composition 612 | Pentadin, purified water, lactic acid |
| Composition 613 | Pentadin, distilled water |
| Composition 614 | Pentadin, distilled water, sodium benzoate, citric, acid |
| Composition 615 | Pentadin, distilled water, lactic acid |
| Composition 616 | Pentadin, vapor distilled electrolyte enhanced water |
| Composition 617 | Pentadin, vapor distilled electrolyte enhanced water, sodium |
| | benzoate, citric acid |
| Composition 618 | Pentadin, vapor distilled electrolyte enhanced water, lactic acid |
| Composition 619 | Pentadin, triple filtered water |
| Composition 620 | Pentadin, triple filtered water, sodium benzoate, citric acid |
| Composition 621 | Pentadin, triple filtered water, lactic acid |
| Composition 622 | Sorbitol, water |
| Composition 623 | Sorbitol, water, sodium benzoate, citric acid |
| Composition 624 | Sorbitol, water, lactic acid |
| Composition 625 | Sorbitol, mineral water |
| Composition 626 | Sorbitol, mineral water, sodium benzoate, citric acid |
| Composition 627 | Sorbitol, mineral water, lactic acid |
| Composition 628 | Sorbitol, natural spring water |
| Composition 629 | Sorbitol, natural spring water, sodium benzoate, citric acid |
| Composition 630 | Sorbitol, natural spring water, lactic acid |
| Composition 631 | Sorbitol, filtered water |
| Composition 632 | Sorbitol, filtered water, sodium benzoate, citric |
| Composition 633 | Sorbitol, filtered water, lactic acid |
| Composition 634 | Sorbitol, artesian water |
| Composition 635 | Sorbitol, artesian water, sodium benzoate, citric acid |
| Composition 636 | Sorbitol, artesian water, lactic acid |
| Composition 637 | Sorbitol, purified water |
| Composition 638 | Sorbitol, purified water, sodium benzoate, citric acid |
| Composition 639 | Sorbitol, purified water, lactic acid |
| Composition 640 | Sorbitol, distilled water |
| Composition 641 | Sorbitol, distilled water, sodium benzoate, citric, acid |
| Composition 642 | Sorbitol, distilled water, lactic acid |
| Composition 643 | Sorbitol, vapor distilled electrolyte enhanced water |
| Composition 644 | Sorbitol, vapor distilled electrolyte enhanced water, sodium |
| | benzoate, citric acid |
| Composition 645 | Sorbitol, vapor distilled electrolyte enhanced water, lactic acid |
| Composition 646 | Sorbitol, triple filtered water |
| Composition 647 | Sorbitol, triple filtered water, sodium benzoate, citric acid |
| Composition 648 | Sorbitol, triple filtered water, lactic acid |
| Composition 649 | Tagatose, water |
| Composition 650 | Tagatose, water, sodium benzoate, citric acid |
| Composition 651 | Tagatose, water, lactic acid |
| Composition 652 | Tagatose, mineral water |
| Composition 653 | Tagatose, mineral water, sodium benzoate, citric acid |
| Composition 654 | Tagatose, mineral water, lactic acid |
| Composition 655 | Tagatose, natural spring water |
| Composition 656 | Tagatose, natural spring water, sodium benzoate, citric acid |
| Composition 657 | Tagatose, natural spring water, lactic acid |
| Composition 658 | Tagatose, filtered water |
| Composition 659 | Tagatose, filtered water, sodium benzoate, citric |
| Composition 660 | Tagatose, filtered water, lactic acid |
| Composition 661 | Tagatose, artesian water |
| Composition 662 | Tagatose, artesian water, sodium benzoate, citric acid |
| Composition 663 | Tagatose, artesian water, lactic acid |
| Composition 664 | Tagatose, purified water |
| Composition 665 | Tagatose, purified water, sodium benzoate, citric acid |
| Composition 666 | Tagatose, purified water, lactic acid |
| Composition 667 | Tagatose, distilled water |
| Composition 668 | Tagatose, distilled water, sodium benzoate, citric, acid |
| Composition 669 | Tagatose, distilled water, lactic acid |
| Composition 670 | Tagatose, vapor distilled electrolyte enhanced water |
| Composition 671 | Tagatose, vapor distilled electrolyte enhanced water, sodium |
| | benzoate, citric acid |
| Composition 672 | Tagatose, vapor distilled electrolyte enhanced water, lactic acid |
| Composition 673 | Tagatose, triple filtered water |
| Composition 674 | Tagatose, triple filtered water, sodium benzoate, citric acid |
| Composition 675 | Tagatose, triple filtered water, lactic acid |
| Composition 676 | Thaumatin, water |
| Composition 677 | Thaumatin, water, sodium benzoate, citric acid |
| Composition 678 | Thaumatin, water, lactic acid |
| Composition 679 | Thaumatin, mineral water |
| Composition 680 | Thaumatin, mineral water, sodium benzoate, citric acid |
| Composition 681 | Thaumatin, mineral water, lactic acid |
| Composition 682 | Thaumatin, natural spring water |
| Composition 683 | Thaumatin, natural spring water, sodium benzoate, citric acid |
| Composition 684 | Thaumatin, natural spring water, lactic acid |
| Composition 685 | Thaumatin, filtered water |
| Composition 686 | Thaumatin, filtered water, sodium benzoate, citric |
| Composition 687 | Thaumatin, filtered water, lactic acid |
| Composition 688 | Thaumatin, artesian water |
| Composition 689 | Thaumatin, artesian water, sodium benzoate, citric acid |
| Composition 690 | Thaumatin, artesian water, lactic acid |
| Composition 691 | Thaumatin, purified water |
| Composition 692 | Thaumatin, purified water, sodium benzoate, citric acid |
| Composition 693 | Thaumatin, purified water, lactic acid |
| Composition 694 | Thaumatin, distilled water |
| Composition 695 | Thaumatin, distilled water, sodium benzoate, citric, acid |
| Composition 696 | Thaumatin, distilled water, lactic acid |
| Composition 697 | Thaumatin, vapor distilled electrolyte enhanced water |
| Composition 698 | Thaumatin, vapor distilled electrolyte enhanced water, sodium |
| | benzoate, citric acid |
| Composition 699 | Thaumatin, vapor distilled electrolyte enhanced water, lactic |
| | acid |
| Composition 700 | Thaumatin, triple filtered water |
| Composition 701 | Thaumatin, triple filtered water, sodium benzoate, citric acid |
| Composition 702 | Thaumatin, triple filtered water, lactic acid |
| Composition 703 | Xylitol, water |
| Composition 704 | Xylitol, water, sodium benzoate, citric acid |
| Composition 705 | Xylitol, water, lactic acid |
| Composition 706 | Xylitol, mineral water |
| Composition 707 | Xylitol, mineral water, sodium benzoate, citric acid |
| Composition 708 | Xylitol, mineral water, lactic acid |
| Composition 709 | Xylitol, natural spring water |
| Composition 710 | Xylitol, natural spring water, sodium benzoate, citric acid |
| Composition 711 | Xylitol, natural spring water, lactic acid |
| Composition 712 | Xylitol, filtered water |
| Composition 713 | Xylitol, filtered water, sodium benzoate, citric |
| Composition 714 | Xylitol, filtered water, lactic acid |
| Composition 715 | Xylitol, artesian water |
| Composition 716 | Xylitol, artesian water, sodium benzoate, citric acid |
| Composition 717 | Xylitol, artesian water, lactic acid |
| Composition 718 | Xylitol, purified water |
| Composition 719 | Xylitol, purified water, sodium benzoate, citric acid |
| Composition 720 | Xylitol, purified water, lactic acid |
| Composition 721 | Xylitol, distilled water |
| Composition 722 | Xylitol, distilled water, sodium benzoate, citric, acid |
| Composition 723 | Xylitol, distilled water, lactic acid |
| Composition 724 | Xylitol, vapor distilled electrolyte enhanced water |
| Composition 725 | Xylitol, vapor distilled electrolyte enhanced water, sodium |
| | benzoate, citric acid |
| Composition 726 | Xylitol, vapor distilled electrolyte enhanced water, lactic acid |
| Composition 727 | Xylitol, triple filtered water |
| Composition 728 | Xylitol, triple filtered water, sodium benzoate, citric acid |
| Composition 729 | Xylitol, triple filtered water, lactic acid |

Although natural sugars and natural sugar substitutes are preferred to formulate the concentrated compositions, compositions with artificial ingredients may be formulated with percentages of artificial ingredients to water measurements that are similar to the embodiments with natural sugars described above (greater than 67.5) or obvious to those skilled in the art based on the disclosure in this application. Artificial sweeteners cannot be measured using the same Brix measurement. Brix being a measure of sugar to water weight, artificial sweeteners have different weight measurements, therefore, they cannot utilize the same method of determining sweetness. However, the sweetening power of artificial compositions is the same, if not, more intense than that of the natural sugar compositions (based upon appropriate ratios of artificial sweetener to water).

Sweetness levels of artificial sweeteners are typically measured by using taste panels. Trained participants on such taste panels are presented with samples of water that have been artificially sweetened to varying degrees. For example, tasters start with plain water. They then drink samples with higher concentrations of sweetness until a taste is detected (which may not be necessarily sweet, just an altered taste). When approximately half of the sample test population detects a change in taste and flavor of the water, flavor chemists establish a “threshold value” for the compound. Based on this threshold value, relative sweetness of a sugar substitute is determined by comparing it to the threshold value of sugar. Sucrose is the threshold for sweetness and flavor that most sugar substitutes try to emulate. By way of example, the general population can detect a solution equivalent to 0.5 (½) percent sucrose (about one teaspoon of sugar) dissolved into 2 cups of water. On the other hand, it takes approximately only one-six-hundredth ( 1/600) of a teaspoon of sucralose (Splenda), to yield the same sweet intensity.

In the embodiments described here, the viscosity levels of compositions with artificial ingredients, should fall somewhere between the viscosity level of water at room temperature of 8.94×10⁻⁴ Pa·s or 0.894 cP and the viscosity level of light corn syrup (which is far too thick but provides a threshold measure for our purposes) of 1.3806 Pa·s or 1380.6 cP. Preferably, the compositions have the viscosity level close to that of an oil (vegetable or olive oil) at a level someplace between 0.080-1.0 Pa·s or 81-1000 cP. Table 3 indicates below various compositions with artificial ingredients.

TABLE 3
COMPOSITIONS WITH ARTIFICIAL SUGAR SUBSTITUTE

| Composition of Liquid | |
|---|---|
| Sugar | Artificial Ingredients |
| Composition 1 | Saccharine, water |
| Composition 2 | Saccharine, water, sodium benzoate, citric acid |
| Composition 3 | Saccharine, water, lactic acid |
| Composition 4 | Saccharine, mineral water |
| Composition 5 | Saccharine, mineral water, sodium benzoate, citric acid |
| Composition 6 | Saccharine, mineral water, lactic acid |
| Composition 7 | Saccharine, natural spring water |
| Composition 8 | Saccharine, natural spring water, sodium benzoate, citric acid |
| Composition 9 | Saccharine, natural spring water, lactic acid |
| Composition 10 | Saccharine, filtered water |
| Composition 11 | Saccharine, filtered water, sodium benzoate, citric |
| Composition 12 | Saccharine, filtered water, lactic acid |
| Composition 13 | Saccharine, artesian water |
| Composition 14 | Saccharine, artesian water, sodium benzoate, citric acid |
| Composition 15 | Saccharine, artesian water, lactic acid |
| Composition 16 | Saccharine, purified water |
| Composition 17 | Saccharine, purified water, sodium benzoate, citric acid |
| Composition 18 | Saccharine, purified water, lactic acid |
| Composition 19 | Saccharine, distilled water |
| Composition 20 | Saccharine, distilled water, sodium benzoate, citric, acid |
| Composition 21 | Saccharine, distilled water, lactic acid |
| Composition 22 | Saccharine, vapor distilled electrolyte enhanced water |
| Composition 23 | Saccharine, vapor distilled electrolyte enhanced water, sodium |
| | benzoate, citric acid |
| Composition 24 | Saccharine, vapor distilled electrolyte enhanced water, lactic |
| | acid |
| Composition 25 | Saccharine, triple filtered water |
| Composition 26 | Saccharine, triple filtered water, sodium benzoate, citric acid |
| Composition 27 | Saccharine, triple filtered water, lactic acid |
| Composition 28 | Aspartame, water |
| Composition 29 | Aspartame, water, sodium benzoate, citric acid |
| Composition 30 | Aspartame, water, lactic acid |
| Composition 31 | Aspartame, mineral water |
| Composition 32 | Aspartame, mineral water, sodium benzoate, citric acid |
| Composition 33 | Aspartame, mineral water, lactic acid |
| Composition 34 | Aspartame, natural spring water |
| Composition 35 | Aspartame, natural spring water, sodium benzoate, citric acid |
| Composition 36 | Aspartame, natural spring water, lactic acid |
| Composition 37 | Aspartame, filtered water |
| Composition 38 | Aspartame, filtered water, sodium benzoate, citric |
| Composition 39 | Aspartame, filtered water, lactic acid |
| Composition 40 | Aspartame, artesian water |
| Composition 41 | Aspartame, artesian water, sodium benzoate, citric acid |
| Composition 42 | Aspartame, artesian water, lactic acid |
| Composition 43 | Aspartame, purified water |
| Composition 44 | Aspartame, purified water, sodium benzoate, citric acid |
| Composition 45 | Aspartame, purified water, lactic acid |
| Composition 46 | Aspartame, distilled water |
| Composition 47 | Aspartame, distilled water, sodium benzoate, citric, acid |
| Composition 48 | Aspartame, distilled water, lactic acid |
| Composition 49 | Aspartame, vapor distilled electrolyte enhanced water |
| Composition 50 | Aspartame, vapor distilled electrolyte enhanced water, sodium |
| | benzoate, citric acid |
| Composition 51 | Aspartame, vapor distilled electrolyte enhanced water, lactic |
| | acid |
| Composition 52 | Aspartame, triple filtered water |
| Composition 53 | Aspartame, triple filtered water, sodium benzoate, citric acid |
| Composition 54 | Aspartame, triple filtered water, lactic acid |
| Composition 55 | Sucralose, water |
| Composition 56 | Sucralose, water, sodium benzoate, citric acid |
| Composition 57 | Sucralose, water, lactic acid |
| Composition 58 | Sucralose, mineral water |
| Composition 59 | Sucralose, mineral water, sodium benzoate, citric acid |
| Composition 60 | Sucralose, mineral water, lactic acid |
| Composition 61 | Sucralose, natural spring water |
| Composition 62 | Sucralose, natural spring water, sodium benzoate, citric acid |
| Composition 63 | Sucralose, natural spring water, lactic acid |
| Composition 64 | Sucralose, filtered water |
| Composition 65 | Sucralose, filtered water, sodium benzoate, citric |
| Composition 66 | Sucralose, filtered water, lactic acid |
| Composition 67 | Sucralose, artesian water |
| Composition 68 | Sucralose, artesian water, sodium benzoate, citric acid |
| Composition 69 | Sucralose, artesian water, lactic acid |
| Composition 70 | Sucralose, purified water |
| Composition 71 | Sucralose, purified water, sodium benzoate, citric acid |
| Composition 72 | Sucralose, purified water, lactic acid |
| Composition 73 | Sucralose, distilled water |
| Composition 74 | Sucralose, distilled water, sodium benzoate, citric, acid |
| Composition 75 | Sucralose, distilled water, lactic acid |
| Composition 76 | Sucralose, vapor distilled electrolyte enhanced water |
| Composition 77 | Sucralose, vapor distilled electrolyte enhanced water, sodium |
| | benzoate, citric acid |
| Composition 78 | Sucralose, vapor distilled electrolyte enhanced water, lactic |
| | acid |
| Composition 79 | Sucralose, triple filtered water |
| Composition 80 | Sucralose, triple filtered water, sodium benzoate, citric acid |
| Composition 81 | Sucralose, triple filtered water, lactic acid |
| Composition 82 | Dextrose, water |
| Composition 83 | Dextrose, water, sodium benzoate, citric acid |
| Composition 84 | Dextrose, water, lactic acid |
| Composition 85 | Dextrose, mineral water |
| Composition 86 | Dextrose, mineral water, sodium benzoate, citric acid |
| Composition 87 | Dextrose, mineral water, lactic acid |
| Composition 88 | Dextrose, natural spring water |
| Composition 89 | Dextrose, natural spring water, sodium benzoate, citric acid |
| Composition 90 | Dextrose, natural spring water, lactic acid |
| Composition 91 | Dextrose, filtered water |
| Composition 92 | Dextrose, filtered water, sodium benzoate, citric |
| Composition 93 | Dextrose, filtered water, lactic acid |
| Composition 94 | Dextrose, artesian water |
| Composition 95 | Dextrose, artesian water, sodium benzoate, citric acid |
| Composition 96 | Dextrose, artesian water, lactic acid |
| Composition 97 | Dextrose, purified water |
| Composition 98 | Dextrose, purified water, sodium benzoate, citric acid |
| Composition 99 | Dextrose, purified water, lactic acid |
| Composition 100 | Dextrose, distilled water |
| Composition 101 | Dextrose, distilled water, sodium benzoate, citric, acid |
| Composition 102 | Dextrose, distilled water, lactic acid |
| Composition 103 | Dextrose, vapor distilled electrolyte enhanced water |
| Composition 104 | Dextrose, vapor distilled electrolyte enhanced water, sodium |
| | benzoate, citric acid |
| Composition 105 | Dextrose, vapor distilled electrolyte enhanced water, lactic |
| | acid |
| Composition 106 | Dextrose, triple filtered water |
| Composition 107 | Dextrose, triple filtered water, sodium benzoate, citric acid |
| Composition 108 | Dextrose, triple filtered water, lactic acid |
| Composition 109 | Isomalt, water |
| Composition 110 | Isomalt, water, sodium benzoate, citric acid |
| Composition 111 | Isomalt, water, lactic acid |
| Composition 112 | Isomalt, mineral water |
| Composition 113 | Isomalt, mineral water, sodium benzoate, citric acid |
| Composition 114 | Isomalt, mineral water, lactic acid |
| Composition 115 | Isomalt, natural spring water |
| Composition 116 | Isomalt, natural spring water, sodium benzoate, citric acid |
| Composition 117 | Isomalt, natural spring water, lactic acid |
| Composition 118 | Isomalt, filtered water |
| Composition 119 | Isomalt, filtered water, sodium benzoate, citric |
| Composition 120 | Isomalt, filtered water, lactic acid |
| Composition 121 | Isomalt, artesian water |
| Composition 122 | Isomalt, artesian water, sodium benzoate, citric acid |
| Composition 123 | Isomalt, artesian water, lactic acid |
| Composition 124 | Isomalt, purified water |
| Composition 125 | Isomalt, purified water, sodium benzoate, citric acid |
| Composition 126 | Isomalt, purified water, lactic acid |
| Composition 127 | Isomalt, distilled water |
| Composition 128 | Isomalt, distilled water, sodium benzoate, citric, acid |
| Composition 129 | Isomalt, distilled water, lactic acid |
| Composition 130 | Isomalt, vapor distilled electrolyte enhanced water |
| Composition 131 | Isomalt, vapor distilled electrolyte enhanced water, sodium |
| | benzoate, citric acid |
| Composition 132 | Isomalt, vapor distilled electrolyte enhanced water, lactic acid |
| Composition 133 | Isomalt, triple filtered water |
| Composition 134 | Isomalt, triple filtered water, sodium benzoate, citric acid |
| Composition 135 | Isomalt, triple filtered water, lactic acid |
| Composition 136 | Mannitol, water |
| Composition 137 | Mannitol, water, sodium benzoate, citric acid |
| Composition 138 | Mannitol, water, lactic acid |
| Composition 139 | Mannitol, mineral water |
| Composition 140 | Mannitol, mineral water, sodium benzoate, citric acid |
| Composition 141 | Mannitol, mineral water, lactic acid |
| Composition 142 | Mannitol, natural spring water |
| Composition 143 | Mannitol, natural spring water, sodium benzoate, citric acid |
| Composition 144 | Mannitol, natural spring water, lactic acid |
| Composition 145 | Mannitol, filtered water |
| Composition 146 | Mannitol, filtered water, sodium benzoate, citric |
| Composition 147 | Mannitol, filtered water, lactic acid |
| Composition 148 | Mannitol, artesian water |
| Composition 149 | Mannitol, artesian water, sodium benzoate, citric acid |
| Composition 150 | Mannitol, artesian water, lactic acid |
| Composition 151 | Mannitol, purified water |
| Composition 152 | Mannitol, purified water, sodium benzoate, citric acid |
| Composition 153 | Mannitol, purified water, lactic acid |
| Composition 154 | Mannitol, distilled water |
| Composition 155 | Mannitol, distilled water, sodium benzoate, citric, acid |
| Composition 156 | Mannitol, distilled water, lactic acid |
| Composition 157 | Mannitol, vapor distilled electrolyte enhanced water |
| Composition 158 | Mannitol, vapor distilled electrolyte enhanced water, sodium |
| | benzoate, citric acid |
| Composition 159 | Mannitol, vapor distilled electrolyte enhanced water, lactic |
| | acid |
| Composition 160 | Mannitol, triple filtered water |
| Composition 161 | Mannitol, triple filtered water, sodium benzoate, citric acid |
| Composition 162 | Mannitol, triple filtered water, lactic acid |
| Composition 163 | Trehalose, water |
| Composition 164 | Trehalose, water, sodium benzoate, citric acid |
| Composition 165 | Trehalose, water, lactic acid |
| Composition 166 | Trehalose, mineral water |
| Composition 167 | Trehalose, mineral water, sodium benzoate, citric acid |
| Composition 168 | Trehalose, mineral water, lactic acid |
| Composition 169 | Trehalose, natural spring water |
| Composition 170 | Trehalose, natural spring water, sodium benzoate, citric acid |
| Composition 171 | Trehalose, natural spring water, lactic acid |
| Composition 172 | Trehalose, filtered water |
| Composition 173 | Trehalose, filtered water, sodium benzoate, citric |
| Composition 174 | Trehalose, filtered water, lactic acid |
| Composition 175 | Trehalose, artesian water |
| Composition 176 | Trehalose, artesian water, sodium benzoate, citric acid |
| Composition 177 | Trehalose, artesian water, lactic acid |
| Composition 178 | Trehalose, purified water |
| Composition 179 | Trehalose, purified water, sodium benzoate, citric acid |
| Composition 180 | Trehalose, purified water, lactic acid |
| Composition 181 | Trehalose, distilled water |
| Composition 182 | Trehalose, distilled water, sodium benzoate, citric, acid |
| Composition 183 | Trehalose, distilled water, lactic acid |
| Composition 184 | Trehalose, vapor distilled electrolyte enhanced water |
| Composition 185 | Trehalose, vapor distilled electrolyte enhanced water, sodium |
| | benzoate, citric acid |
| Composition 186 | Trehalose, vapor distilled electrolyte enhanced water, lactic |
| | acid |
| Composition 187 | Trehalose, triple filtered water |
| Composition 188 | Trehalose, triple filtered water, sodium benzoate, citric acid |
| Composition 189 | Trehalose, triple filtered water, lactic acid |
| Composition 190 | Sucromalt, water |
| Composition 191 | Sucromalt, water, sodium benzoate, citric acid |
| Composition 192 | Sucromalt, water, lactic acid |
| Composition 193 | Sucromalt, mineral water |
| Composition 194 | Sucromalt, mineral water, sodium benzoate, citric acid |
| Composition 195 | Sucromalt, mineral water, lactic acid |
| Composition 196 | Sucromalt, natural spring water |
| Composition 197 | Sucromalt, natural spring water, sodium benzoate, citric acid |
| Composition 198 | Sucromalt, natural spring water, lactic acid |
| Composition 199 | Sucromalt, filtered water |
| Composition 200 | Sucromalt, filtered water, sodium benzoate, citric |
| Composition 201 | Sucromalt, filtered water, lactic acid |
| Composition 202 | Sucromalt, artesian water |
| Composition 203 | Sucromalt, artesian water, sodium benzoate, citric acid |
| Composition 204 | Sucromalt, artesian water, lactic acid |
| Composition 205 | Sucromalt, purified water |
| Composition 206 | Sucromalt, purified water, sodium benzoate, citric acid |
| Composition 207 | Sucromalt, purified water, lactic acid |
| Composition 208 | Sucromalt, distilled water |
| Composition 209 | Sucromalt, distilled water, sodium benzoate, citric, acid |
| Composition 210 | Sucromalt, distilled water, lactic acid |
| Composition 211 | Sucromalt, vapor distilled electrolyte enhanced water |
| Composition 212 | Sucromalt, vapor distilled electrolyte enhanced water, sodium |
| | benzoate, citric acid |
| Composition 213 | Sucromalt, vapor distilled electrolyte enhanced water, lactic |
| | acid |
| Composition 214 | Sucromalt, triple filtered water |
| Composition 215 | Sucromalt, triple filtered water, sodium benzoate, citric acid |
| Composition 216 | Sucromalt, triple filtered water, lactic acid |
| Composition 217 | Maltitol, water |
| Composition 218 | Maltitol, water, sodium benzoate, citric acid |
| Composition 219 | Maltitol, water, lactic acid |
| Composition 220 | Maltitol, mineral water |
| Composition 221 | Maltitol, mineral water, sodium benzoate, citric acid |
| Composition 222 | Maltitol, mineral water, lactic acid |
| Composition 223 | Maltitol, natural spring water |
| Composition 224 | Maltitol, natural spring water, sodium benzoate, citric acid |
| Composition 225 | Maltitol, natural spring water, lactic acid |
| Composition 226 | Maltitol, filtered water |
| Composition 227 | Maltitol, filtered water, sodium benzoate, citric |
| Composition 228 | Maltitol, filtered water, lactic acid |
| Composition 229 | Maltitol, artesian water |
| Composition 230 | Maltitol, artesian water, sodium benzoate, citric acid |
| Composition 231 | Maltitol, artesian water, lactic acid |
| Composition 232 | Maltitol, purified water |
| Composition 233 | Maltitol, purified water, sodium benzoate, citric acid |
| Composition 234 | Maltitol, purified water, lactic acid |
| Composition 235 | Maltitol, distilled water |
| Composition 236 | Maltitol, distilled water, sodium benzoate, citric, acid |
| Composition 237 | Maltitol, distilled water, lactic acid |
| Composition 238 | Maltitol, vapor distilled electrolyte enhanced water |
| Composition 239 | Maltitol, vapor distilled electrolyte enhanced water, sodium |
| | benzoate, citric acid |
| Composition 240 | Maltitol, vapor distilled electrolyte enhanced water, lactic acid |
| Composition 241 | Maltitol, triple filtered water |
| Composition 242 | Maltitol, triple filtered water, sodium benzoate, citric acid |
| Composition 243 | Maltitol, triple filtered water, lactic acid |
| Composition 244 | Sorbitol, water |
| Composition 245 | Sorbitol, water, sodium benzoate, citric acid |
| Composition 246 | Sorbitol, water, lactic acid |
| Composition 247 | Sorbitol, mineral water |
| Composition 248 | Sorbitol, mineral water, sodium benzoate, citric acid |
| Composition 249 | Sorbitol, mineral water, lactic acid |
| Composition 250 | Sorbitol, natural spring water |
| Composition 251 | Sorbitol, natural spring water, sodium benzoate, citric acid |
| Composition 252 | Sorbitol, natural spring water, lactic acid |
| Composition 253 | Sorbitol, filtered water |
| Composition 254 | Sorbitol, filtered water, sodium benzoate, citric |
| Composition 255 | Sorbitol, filtered water, lactic acid |
| Composition 256 | Sorbitol, artesian water |
| Composition 257 | Sorbitol, artesian water, sodium benzoate, citric acid |
| Composition 258 | Sorbitol, artesian water, lactic acid |
| Composition 259 | Sorbitol, purified water |
| Composition 260 | Sorbitol, purified water, sodium benzoate, citric acid |
| Composition 261 | Sorbitol, purified water, lactic acid |
| Composition 262 | Sorbitol, distilled water |
| Composition 263 | Sorbitol, distilled water, sodium benzoate, citric, acid |
| Composition 264 | Sorbitol, distilled water, lactic acid |
| Composition 265 | Sorbitol, vapor distilled electrolyte enhanced water |
| Composition 266 | Sorbitol, vapor distilled electrolyte enhanced water, sodium |
| | benzoate, citric acid |
| Composition 267 | Sorbitol, vapor distilled electrolyte enhanced water, lactic acid |
| Composition 268 | Sorbitol, triple filtered water |
| Composition 269 | Sorbitol, triple filtered water, sodium benzoate, citric acid |
| Composition 270 | Sorbitol, triple filtered water, lactic acid |
| Composition 271 | Xylitol, water |
| Composition 272 | Xylitol, water, sodium benzoate, citric acid |
| Composition 273 | Xylitol, water, lactic acid |
| Composition 274 | Xylitol, mineral water |
| Composition 275 | Xylitol, mineral water, sodium benzoate, citric acid |
| Composition 276 | Xylitol, mineral water, lactic acid |
| Composition 277 | Xylitol, natural spring water |
| Composition 278 | Xylitol, natural spring water, sodium benzoate, citric acid |
| Composition 279 | Xylitol, natural spring water, lactic acid |
| Composition 280 | Xylitol, filtered water |
| Composition 281 | Xylitol, filtered water, sodium benzoate, citric |
| Composition 282 | Xylitol, filtered water, lactic acid |
| Composition 283 | Xylitol, artesian water |
| Composition 284 | Xylitol, artesian water, sodium benzoate, citric acid |
| Composition 285 | Xylitol, artesian water, lactic acid |
| Composition 286 | Xylitol, purified water |
| Composition 287 | Xylitol, purified water, sodium benzoate, citric acid |
| Composition 288 | Xylitol, purified water, lactic acid |
| Composition 289 | Xylitol, distilled water |
| Composition 290 | Xylitol, distilled water, sodium benzoate, citric, acid |
| Composition 291 | Xylitol, distilled water, lactic acid |
| Composition 292 | Xylitol, vapor distilled electrolyte enhanced water |
| Composition 293 | Xylitol, vapor distilled electrolyte enhanced water, sodium |
| | benzoate, citric acid |
| Composition 294 | Xylitol, vapor distilled electrolyte enhanced water, lactic acid |
| Composition 295 | Xylitol, triple filtered water |
| Composition 296 | Xylitol, triple filtered water, sodium benzoate, citric acid |
| Composition 297 | Xylitol, triple filtered water, lactic acid |
| Composition 298 | Neotame, water |
| Composition 299 | Neotame, water, sodium benzoate, citric acid |
| Composition 300 | Neotame, water, lactic acid |
| Composition 301 | Neotame, mineral water |
| Composition 302 | Neotame, mineral water, sodium benzoate, citric acid |
| Composition 303 | Neotame, mineral water, lactic acid |
| Composition 304 | Neotame, natural spring water |
| Composition 305 | Neotame, natural spring water, sodium benzoate, citric acid |
| Composition 306 | Neotame, natural spring water, lactic acid |
| Composition 307 | Neotame, filtered water |
| Composition 308 | Neotame, filtered water, sodium benzoate, citric |
| Composition 309 | Neotame, filtered water, lactic acid |
| Composition 310 | Neotame, artesian water |
| Composition 311 | Neotame, artesian water, sodium benzoate, citric acid |
| Composition 312 | Neotame, artesian water, lactic acid |
| Composition 313 | Neotame, purified water |
| Composition 314 | Neotame, purified water, sodium benzoate, citric acid |
| Composition 315 | Neotame, purified water, lactic acid |
| Composition 316 | Neotame, distilled water |
| Composition 317 | Neotame, distilled water, sodium benzoate, citric, acid |
| Composition 318 | Neotame, distilled water, lactic acid |
| Composition 319 | Neotame, vapor distilled electrolyte enhanced water |
| Composition 320 | Neotame, vapor distilled electrolyte enhanced water, sodium |
| | benzoate, citric acid |
| Composition 321 | Neotame, vapor distilled electrolyte enhanced water, lactic |
| | acid |
| Composition 322 | Neotame, triple filtered water |
| Composition 323 | Neotame, triple filtered water, sodium benzoate, citric acid |
| Composition 324 | Neotame, triple filtered water, lactic acid |
| Composition 325 | Acesulfame-K, water |
| Composition 326 | Acesulfame-K, water, sodium benzoate, citric acid |
| Composition 327 | Acesulfame-K, water, lactic acid |
| Composition 328 | Acesulfame-K, mineral water |
| Composition 329 | Acesulfame-K, mineral water, sodium benzoate, citric acid |
| Composition 330 | Acesulfame-K, mineral water, lactic acid |
| Composition 331 | Acesulfame-K, natural spring water |
| Composition 332 | Acesulfame-K, natural spring water, sodium benzoate, citric |
| | acid |
| Composition 333 | Acesulfame-K, natural spring water, lactic acid |
| Composition 334 | Acesulfame-K, filtered water |
| Composition 335 | Acesulfame-K, filtered water, sodium benzoate, citric |
| Composition 336 | Acesulfame-K, filtered water, lactic acid |
| Composition 337 | Acesulfame-K, artesian water |
| Composition 338 | Acesulfame-K, artesian water, sodium benzoate, citric acid |
| Composition 339 | Acesulfame-K, artesian water, lactic acid |
| Composition 340 | Acesulfame-K, purified water |
| Composition 341 | Acesulfame-K, purified water, sodium benzoate, citric acid |
| Composition 342 | Acesulfame-K, purified water, lactic acid |
| Composition 343 | Acesulfame-K, distilled water |
| Composition 344 | Acesulfame-K, distilled water, sodium benzoate, citric, acid |
| Composition 345 | Acesulfame-K, distilled water, lactic acid |
| Composition 346 | Acesulfame-K, vapor distilled electrolyte enhanced water |
| Composition 347 | Acesulfame-K, vapor distilled electrolyte enhanced water, |
| | sodium benzoate, citric acid |
| Composition 348 | Acesulfame-K, vapor distilled electrolyte enhanced water, |
| | lactic acid |
| Composition 349 | Acesulfame-K, triple filtered water |
| Composition 350 | Acesulfame-K, triple filtered water, sodium benzoate, citric |
| | acid |
| Composition 351 | Acesulfame-K, triple filtered water, lactic acid |
| Composition 352 | Alitame, water |
| Composition 353 | Alitame, water, sodium benzoate, citric acid |
| Composition 354 | Alitame, water, lactic acid |
| Composition 355 | Alitame, mineral water |
| Composition 356 | Alitame, mineral water, sodium benzoate, citric acid |
| Composition 357 | Alitame, mineral water, lactic acid |
| Composition 358 | Alitame, natural spring water |
| Composition 359 | Alitame, natural spring water, sodium benzoate, citric acid |
| Composition 360 | Alitame, natural spring water, lactic acid |
| Composition 361 | Alitame, filtered water |
| Composition 362 | Alitame, filtered water, sodium benzoate, citric |
| Composition 363 | Alitame, filtered water, lactic acid |
| Composition 364 | Alitame, artesian water |
| Composition 365 | Alitame, artesian water, sodium benzoate, citric acid |
| Composition 366 | Alitame, artesian water, lactic acid |
| Composition 367 | Alitame, purified water |
| Composition 368 | Alitame, purified water, sodium benzoate, citric acid |
| Composition 369 | Alitame, purified water, lactic acid |
| Composition 370 | Alitame, distilled water |
| Composition 371 | Alitame, distilled water, sodium benzoate, citric, acid |
| Composition 372 | Alitame, distilled water, lactic acid |
| Composition 373 | Alitame, vapor distilled electrolyte enhanced water |
| Composition 374 | Alitame, vapor distilled electrolyte enhanced water, sodium |
| | benzoate, citric acid |
| Composition 375 | Alitame, vapor distilled electrolyte enhanced water, lactic acid |
| Composition 376 | Alitame, triple filtered water |
| Composition 377 | Alitame, triple filtered water, sodium benzoate, citric acid |
| Composition 378 | Alitame, triple filtered water, lactic acid |
| Composition 379 | Cyclamate, water |
| Composition 380 | Cyclamate, water, sodium benzoate, citric acid |
| Composition 381 | Cyclamate, water, lactic acid |
| Composition 382 | Cyclamate, mineral water |
| Composition 383 | Cyclamate, mineral water, sodium benzoate, citric acid |
| Composition 384 | Cyclamate, mineral water, lactic acid |
| Composition 385 | Cyclamate, natural spring water |
| Composition 386 | Cyclamate, natural spring water, sodium benzoate, citric acid |
| Composition 387 | Cyclamate, natural spring water, lactic acid |
| Composition 388 | Cyclamate, filtered water |
| Composition 389 | Cyclamate, filtered water, sodium benzoate, citric |
| Composition 390 | Cyclamate, filtered water, lactic acid |
| Composition 391 | Cyclamate, artesian water |
| Composition 392 | Cyclamate, artesian water, sodium benzoate, citric acid |
| Composition 393 | Cyclamate, artesian water, lactic acid |
| Composition 394 | Cyclamate, purified water |
| Composition 395 | Cyclamate, purified water, sodium benzoate, citric acid |
| Composition 396 | Cyclamate, purified water, lactic acid |
| Composition 397 | Cyclamate, distilled water |
| Composition 398 | Cyclamate, distilled water, sodium benzoate, citric, acid |
| Composition 399 | Cyclamate, distilled water, lactic acid |
| Composition 400 | Cyclamate, vapor distilled electrolyte enhanced water |
| Composition 401 | Cyclamate, vapor distilled electrolyte enhanced water, sodium |
| | benzoate, citric acid |
| Composition 402 | Cyclamate, vapor distilled electrolyte enhanced water, lactic |
| | acid |
| Composition 403 | Cyclamate, triple filtered water |
| Composition 404 | Cyclamate, triple filtered water, sodium benzoate, citric acid |
| Composition 405 | Cyclamate, triple filtered water, lactic acid |
| Composition 406 | Acesulfame potassium, water |
| Composition 407 | Acesulfame potassium, water, sodium benzoate, citric acid |
| Composition 408 | Acesulfame potassium, water, lactic acid |
| Composition 409 | Acesulfame potassium, mineral water |
| Composition 410 | Acesulfame potassium, mineral water, sodium benzoate, citric |
| | acid |
| Composition 411 | Acesulfame potassium, mineral water, lactic acid |
| Composition 412 | Acesulfame potassium, natural spring water |
| Composition 413 | Acesulfame potassium, natural spring water, sodium benzoate, |
| | citric acid |
| Composition 414 | Acesulfame potassium, natural spring water, lactic acid |
| Composition 415 | Acesulfame potassium, filtered water |
| Composition 416 | Acesulfame potassium, filtered water, sodium benzoate, citric |
| Composition 417 | Acesulfame potassium, filtered water, lactic acid |
| Composition 418 | Acesulfame potassium, artesian water |
| Composition 419 | Acesulfame potassium, artesian water, sodium benzoate, citric |
| | acid |
| Composition 420 | Acesulfame potassium, artesian water, lactic acid |
| Composition 421 | Acesulfame potassium, purified water |
| Composition 422 | Acesulfame potassium, purified water, sodium benzoate, citric |
| | acid |
| Composition 423 | Acesulfame potassium, purified water, lactic acid |
| Composition 424 | Acesulfame potassium, distilled water |
| Composition 425 | Acesulfame potassium, distilled water, sodium benzoate, citric, |
| | acid |
| Composition 426 | Acesulfame potassium, distilled water, lactic acid |
| Composition 427 | Acesulfame potassium, vapor distilled electrolyte enhanced |
| | water |
| Composition 428 | Acesulfame potassium, vapor distilled electrolyte enhanced |
| | water, sodium benzoate, citric acid |
| Composition 429 | Acesulfame potassium, vapor distilled electrolyte enhanced |
| | water, lactic acid |
| Composition 430 | Acesulfame potassium, triple filtered water |
| Composition 431 | Acesulfame potassium, triple filtered water, sodium benzoate, |
| | citric acid |
| Composition 432 | Acesulfame potassium, triple filtered water, lactic acid |
| Composition 433 | Alitame, water |
| Composition 434 | Alitame, water, sodium benzoate, citric acid |
| Composition 435 | Alitame, water, lactic acid |
| Composition 436 | Alitame, mineral water |
| Composition 437 | Alitame, mineral water, sodium benzoate, citric acid |
| Composition 438 | Alitame, mineral water, lactic acid |
| Composition 439 | Alitame, natural spring water |
| Composition 440 | Alitame, natural spring water, sodium benzoate, citric acid |
| Composition 441 | Alitame, natural spring water, lactic acid |
| Composition 442 | Alitame, filtered water |
| Composition 443 | Alitame, filtered water, sodium benzoate, citric |
| Composition 445 | Alitame, filtered water, lactic acid |
| Composition 446 | Alitame, artesian water |
| Composition 447 | Alitame, artesian water, sodium benzoate, citric acid |
| Composition 448 | Alitame, artesian water, lactic acid |
| Composition 449 | Alitame, purified water |
| Composition 450 | Alitame, purified water, sodium benzoate, citric acid |
| Composition 451 | Alitame, purified water, lactic acid |
| Composition 452 | Alitame, distilled water |
| Composition 453 | Alitame, distilled water, sodium benzoate, citric, acid |
| Composition 454 | Alitame, distilled water, lactic acid |
| Composition 455 | Alitame, vapor distilled electrolyte enhanced water |
| Composition 456 | Alitame, vapor distilled electrolyte enhanced water, sodium |
| | benzoate, citric acid |
| Composition 457 | Alitame, vapor distilled electrolyte enhanced water, lactic acid |
| Composition 458 | Alitame, triple filtered water |
| Composition 459 | Alitame, triple filtered water, sodium benzoate, citric acid |
| Composition 460 | Alitame, triple filtered water, lactic acid |
| Composition 461 | Salt of aspartame-acesulfame, water |
| Composition 462 | Salt of aspartame-acesulfame, water, sodium benzoate, citric |
| | acid |
| Composition 463 | Salt of aspartame-acesulfame, water, lactic acid |
| Composition 464 | Salt of aspartame-acesulfame, mineral water |
| Composition 465 | Salt of aspartame-acesulfame, mineral water, sodium benzoate, |
| | citric acid |
| Composition 466 | Salt of aspartame-acesulfame, mineral water, lactic acid |
| Composition 467 | Salt of aspartame-acesulfame, natural spring water |
| Composition 468 | Salt of aspartame-acesulfame, natural spring water, sodium |
| | benzoate, citric acid |
| Composition 469 | Salt of aspartame-acesulfame, natural spring water, lactic acid |
| Composition 470 | Salt of aspartame-acesulfame, filtered water |
| Composition 471 | Salt of aspartame-acesulfame, filtered water, sodium benzoate, |
| | citric |
| Composition 472 | Salt of aspartame-acesulfame, filtered water, lactic acid |
| Composition 473 | Salt of aspartame-acesulfame, artesian water |
| Composition 474 | Salt of aspartame-acesulfame, artesian water, sodium |
| | benzoate, citric acid |
| Composition 475 | Salt of aspartame-acesulfame, artesian water, lactic acid |
| Composition 476 | Salt of aspartame-acesulfame, purified water |
| Composition 477 | Salt of aspartame-acesulfame, purified water, sodium |
| | benzoate, citric acid |
| Composition 478 | Salt of aspartame-acesulfame, purified water, lactic acid |
| Composition 479 | Salt of aspartame-acesulfame, distilled water |
| Composition 480 | Salt of aspartame-acesulfame, distilled water, sodium |
| | benzoate, citric, acid |
| Composition 481 | Salt of aspartame-acesulfame, distilled water, lactic acid |
| Composition 482 | Salt of aspartame-acesulfame, vapor distilled electrolyte |
| | enhanced water |
| Composition 483 | Salt of aspartame-acesulfame, vapor distilled electrolyte |
| | enhanced water, sodium benzoate, citric acid |
| Composition 484 | Salt of aspartame-acesulfame, vapor distilled electrolyte |
| | enhanced water, lactic acid |
| Composition 485 | Salt of aspartame-acesulfame, triple filtered water |
| Composition 486 | Salt of aspartame-acesulfame, triple filtered water, sodium |
| | benzoate, citric acid |
| Composition 487 | Salt of aspartame-acesulfame, triple filtered water, lactic acid |
| Composition 488 | Dulcin, water |
| Composition 489 | Dulcin, water, sodium benzoate, citric acid |
| Composition 490 | Dulcin, water, lactic acid |
| Composition 491 | Dulcin, mineral water |
| Composition 492 | Dulcin, mineral water, sodium benzoate, citric acid |
| Composition 493 | Dulcin, mineral water, lactic acid |
| Composition 494 | Dulcin, natural spring water |
| Composition 495 | Dulcin, natural spring water, sodium benzoate, citric acid |
| Composition 496 | Dulcin, natural spring water, lactic acid |
| Composition 497 | Dulcin, filtered water |
| Composition 498 | Dulcin, filtered water, sodium benzoate, citric |
| Composition 499 | Dulcin, filtered water, lactic acid |
| Composition 500 | Dulcin, artesian water |
| Composition 501 | Dulcin, artesian water, sodium benzoate, citric acid |
| Composition 502 | Dulcin, artesian water, lactic acid |
| Composition 503 | Dulcin, purified water |
| Composition 504 | Dulcin, purified water, sodium benzoate, citric acid |
| Composition 505 | Dulcin, purified water, lactic acid |
| Composition 506 | Dulcin, distilled water |
| Composition 507 | Dulcin, distilled water, sodium benzoate, citric, acid |
| Composition 508 | Dulcin, distilled water, lactic acid |
| Composition 509 | Dulcin, vapor distilled electrolyte enhanced water |
| Composition 510 | Dulcin, vapor distilled electrolyte enhanced water, sodium |
| | benzoate, citric acid |
| Composition 511 | Dulcin, vapor distilled electrolyte enhanced water, lactic acid |
| Composition 512 | Dulcin, triple filtered water |
| Composition 513 | Dulcin, triple filtered water, sodium benzoate, citric acid |
| Composition 514 | Dulcin, triple filtered water, lactic acid |
| Composition 515 | Glucin, water |
| Composition 516 | Glucin, water, sodium benzoate, citric acid |
| Composition 517 | Glucin, water, lactic acid |
| Composition 518 | Glucin, mineral water |
| Composition 519 | Glucin, mineral water, sodium benzoate, citric acid |
| Composition 520 | Glucin, mineral water, lactic acid |
| Composition 521 | Glucin, natural spring water |
| Composition 522 | Glucin, natural spring water, sodium benzoate, citric acid |
| Composition 523 | Glucin, natural spring water, lactic acid |
| Composition 524 | Glucin, filtered water |
| Composition 525 | Glucin, filtered water, sodium benzoate, citric |
| Composition 526 | Glucin, filtered water, lactic acid |
| Composition 527 | Glucin, artesian water |
| Composition 528 | Glucin, artesian water, sodium benzoate, citric acid |
| Composition 529 | Glucin, artesian water, lactic acid |
| Composition 530 | Glucin, purified water |
| Composition 531 | Glucin, purified water, sodium benzoate, citric acid |
| Composition 532 | Glucin, purified water, lactic acid |
| Composition 533 | Glucin, distilled water |
| Composition 534 | Glucin, distilled water, sodium benzoate, citric, acid |
| Composition 535 | Glucin, distilled water, lactic acid |
| Composition 536 | Glucin, vapor distilled electrolyte enhanced water |
| Composition 537 | Glucin, vapor distilled electrolyte enhanced water, sodium |
| | benzoate, citric acid |
| Composition 538 | Glucin, vapor distilled electrolyte enhanced water, lactic acid |
| Composition 539 | Glucin, triple filtered water |
| Composition 540 | Glucin, triple filtered water, sodium benzoate, citric acid |
| Composition 541 | Glucin, triple filtered water, lactic acid |
| Composition 542 | Neohesperidin dihydrochalcone, water |
| Composition 543 | Neohesperidin dihydrochalcone, water, sodium benzoate, |
| | citric acid |
| Composition 544 | Neohesperidin dihydrochalcone, water, lactic acid |
| Composition 545 | Neohesperidin dihydrochalcone, mineral water |
| Composition 546 | Neohesperidin dihydrochalcone, mineral water, sodium |
| | benzoate, citric acid |
| Composition 547 | Neohesperidin dihydrochalcone, mineral water, lactic acid |
| Composition 548 | Neohesperidin dihydrochalcone, natural spring water |
| Composition 549 | Neohesperidin dihydrochalcone, natural spring water, sodium |
| | benzoate, citric acid |
| Composition 550 | Neohesperidin dihydrochalcone, natural spring water, lactic |
| | acid |
| Composition 551 | Neohesperidin dihydrochalcone, filtered water |
| Composition 552 | Neohesperidin dihydrochalcone, filtered water, sodium |
| | benzoate, citric |
| Composition 553 | Neohesperidin dihydrochalcone, filtered water, lactic acid |
| Composition 554 | Neohesperidin dihydrochalcone, artesian water |
| Composition 555 | Neohesperidin dihydrochalcone, artesian water, sodium |
| | benzoate, citric acid |
| Composition 556 | Neohesperidin dihydrochalcone, artesian water, lactic acid |
| Composition 557 | Neohesperidin dihydrochalcone, purified water |
| Composition 558 | Neohesperidin dihydrochalcone, purified water, sodium |
| | benzoate, citric acid |
| Composition 559 | Neohesperidin dihydrochalcone, purified water, lactic acid |
| Composition 560 | Neohesperidin dihydrochalcone, distilled water |
| Composition 561 | Neohesperidin dihydrochalcone, distilled water, sodium |
| | benzoate, citric, acid |
| Composition 562 | Neohesperidin dihydrochalcone, distilled water, lactic acid |
| Composition 563 | Neohesperidin dihydrochalcone, vapor distilled electrolyte |
| | enhanced water |
| Composition 564 | Neohesperidin dihydrochalcone, vapor distilled electrolyte |
| | enhanced water, sodium benzoate, citric acid |
| Composition 565 | Neohesperidin dihydrochalcone, vapor distilled electrolyte |
| | enhanced water, lactic acid |
| Composition 566 | Neohesperidin dihydrochalcone, triple filtered water |
| Composition 567 | Neohesperidin dihydrochalcone, triple filtered water, sodium |
| | benzoate, citric acid |
| Composition 568 | Neohesperidin dihydrochalcone, triple filtered water, lactic |
| | acid |

With reference to Table 4, examples of some natural sugar substitutes and the intensity of their sweetness relative to natural sugar are indicated.

TABLE 4
COMPARISON OF SWEETNESS LEVEL OF NATURAL SUGAR
SUBSTITUTES TO NATURAL SUGAR

| Sweetener | Sweetness Factor |
|---|---|
| Brazzein | Protein, 800x sweetness of sucrose (by weight) |
| Curculin | Protein, 550x sweetness (by weight) |
| Erythritol | 0.7x sweetness (by weight), 14x sweetness of sucrose (by |
| | food energy), 0.05x energy density of sucrose |
| Fructose | 1.7x sweetness (by weight and food energy), 1.0x energy |
| | density of sucrose |
| Glycyrrhizin | 50x sweetness (by weight) |
| Glycerol | 0.6x sweetness (by weight), 0.55x sweetness (by food energy), |
| | 1.075x energy density, E422 |
| Hydrogenated starch | 0.4x-0.9x sweetness (by weight), 0.5x-1.2x sweetness (by |
| hydrolysates | food energy), 0.75x energy density |
| Inulin | |
| Isomalt | 0.45x-0.65x sweetness (by weight), 0.9x-1.3x sweetness (by |
| | food energy), 0.5x energy density, E953 |
| Lactitol | 0.4x sweetness (by weight), 0.8x sweetness (by food energy), |
| | 0.5x energy density, E966 |
| Lo Han Guo | 300x sweetness (by weight) |
| Mabinlin | Protein, 100x sweetness (by weight) |
| Maltitol | 0.9x sweetness (by weight), 1.7x sweetness (by food energy), |
| | 0.525x energy density, E965 |
| Maltooligosaccharide | |
| Mannitol | 0.5x sweetness (by weight), 1.2x sweetness (by food energy), |
| | 0.4x energy density, E421 |
| Miraculin | Protein, does not taste sweet by itself, but modifies taste |
| | receptors to make sour things taste sweet temporarily |
| Monatin | naturally-occurring sweetener isolated from the plant |
| | Sclerochiton ilicifolius |
| Monellin | Protein, 3,000x sweetness (by weight) |
| Pentadin | Protein, 500x sweetness (by weight) |
| Sorbitol | 0.6x sweetness (by weight), 0.9x sweetness (by food energy), |
| | 0.65x energy density, E420 |
| Stevia | 250x sweetness (by weight) - extracts known as rebiana, |
| | Truvia, PureVia; mainly containing rebaudioside A, a steviol |
| | glycoside |
| Tagatose | 0.92x sweetness (by weight), 2.4x sweetness (by food energy), |
| | 0.38x energy density |
| Thaumatin | Protein, 2,000x sweetness (by weight), E957 |
| Xylitol | 1.0x sweetness (by weight), 1.7x sweetness (by food energy), |
| | 0.6x energy density, E967 |

With reference to Table 5, examples of some artificial sugar substitutes and the intensity of their sweetness relative to natural sugar are indicated.

TABLE 5
COMPARISON OF SWEETNESS OF ARTIFICIAL SUGAR
SUBSTITUTES TO NATURAL SUGAR

| Artificial Sweetener | Artificial Sweetness Factor |
|---|---|
| Acesulfame potassium | 200x sweetness (by weight), Nutrinova, E950, FDA |
| | Approved 1988 |
| Alitame | 2,000x sweetness (by weight), Pfizer, Pending FDA Approval |
| Aspartame | 160-200x sweetness (by weight), NutraSweet, E951, FDA |
| | Approved 1981 |
| Salt of aspartame-acesulfame | 350x sweetness (by weight), Twinsweet, E962 |
| Cyclamate | 30x sweetness (by weight), Abbott, E952, FDA Banned 1969, |
| | pending re-approval |
| Dulcin | 250x sweetness (by weight), FDA Banned 1950 |
| Glucin | 300x sweetness (by weight) |
| Neohesperidin | 1,500x sweetness (by weight), E959 |
| dihydrochalcone | |
| Neotame | 8,000x sweetness (by weight), NutraSweet, FDA Approved |
| | 2002 |
| P-4000 | 4,000x sweetness (by weight), FDA Banned 1950 |
| Saccharin | 300x sweetness (by weight), E954, FDA Approved 1958 |
| Sucralose | 600x sweetness (by weight), Splenda, Tate & Lyle, E955, |
| | FDA Approved 1998 |

Referring now to FIGS. 6 and 6A, the sachet 100 is formulated with techniques that use improved starch compositions of cross-linked cationic starch, used in the papermaking process, that are eco-friendly. With such techniques, the recyclable and compostable packaging is formed to hold the liquid sugar composition in dry, damp, or wet conditions. Such packaging is typically produced through the use of pregelatinized starch solutions and pregelatinized paper starch incorporating pre-gelled starch suspension that is unique in its ability to form hydrated gels. The packaging may be further coated with any suitable liquid-resistant coating. Examples of such liquid-resistant coatings include, but are not limited to, coatings such as PROTECoat (from New Coat, Inc.), Zein® (a biodegradable material isolated from corn); polylactic acid (PLA, a polymer of lactic acid from fermentation feed stock); poly hydroxyalkanoates (PHA, from microbial fermentation); bacterial cellulose; chitosan based polymers (for example from shell fish wastes); or waxes and oil based coatings. These materials can be applied either as a thin film or can be sprayed/dipped onto the packaging. Bonding processes for application of thin films of water-resistant material are known in the art.

A waterproof coating is desirable for packaging or sachets that are intended to be in contact with water. As the sachets having a starch-based binder have a high affinity for water, the preferred coatings are non-aqueous and have a low polarity. Appropriate coatings include paraffin (synthetic wax); shellac; xylene-formaldehyde resins condensed with 4,4′-isopropylidenediphenolepichlorohydrin epoxy resins; drying oils; reconstituted oils from triglycerides or fatty acids from the drying oils to form esters with various glycols (butylene glycol, ethylene glycol), sorbitol, and trimethylol ethane or propane; synthetic drying oils including polybutadiene resin.

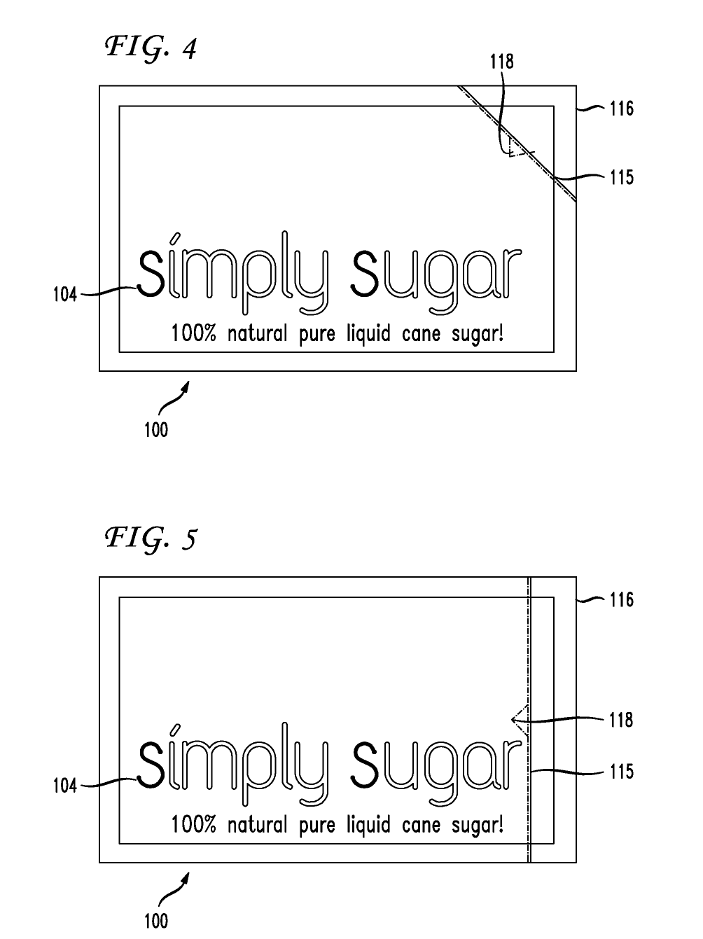

With further reference to FIGS. 4, 5 and 6, the sachet 100 is formed with a perforated edge 115 along any extremity configured to easily detach with slight pressure exerted by a consumer. The perforated edge is rigidly attached to allow one corner 116 to easily detach and reveal a spout-like orifice 118, through which a consumer can easily pour out the liquid sugar composition housed within the packaging or sachet 100.

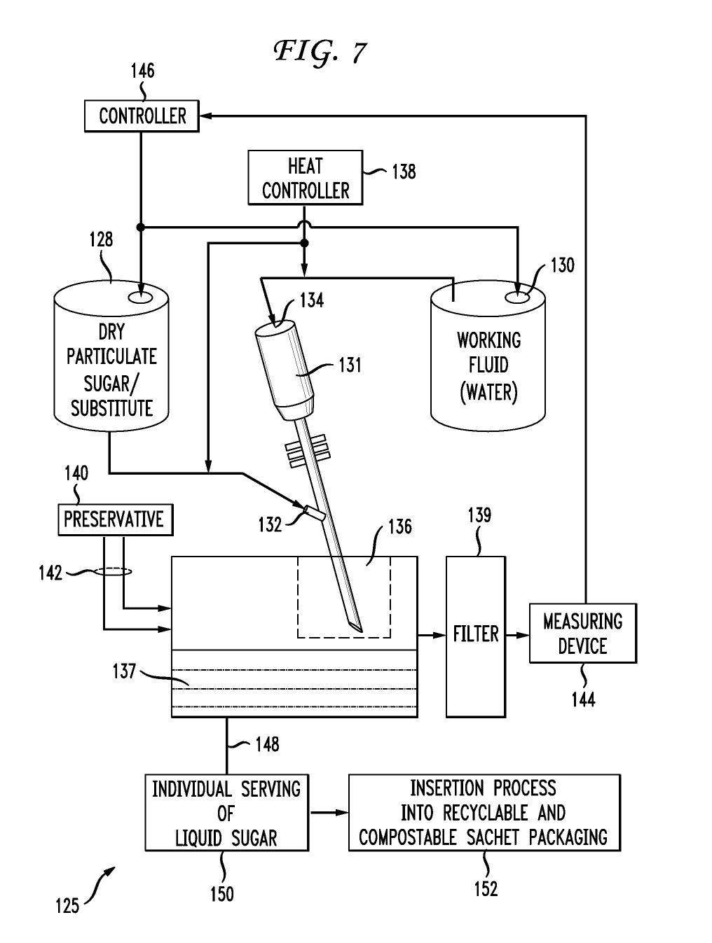

Referring now to FIG. 7, the system in accordance with present invention to formulate the concentrated liquid sugar compositions (disclosed in TABLES 1, 2, and 3) and package it for individual serving in recyclable and compostable sachets is illustrated generally at 125. The system 125 comprises a sugar feed system indicated at 128, which houses dry particulate sugar, and a fluid container 130 for housing a working fluid including water. The various types of sweeteners, natural and artificial are disclosed in TABLES 1, 2, and 3. In the preferred embodiment, natural sweeteners and sugar substitutes are recommended for obvious health reasons. However, artificial sweeteners such as those disclosed in TABLE 3 may also be used. The type of water may be any one of the ones specified in TABLES 1, 2, and 3 or other types used in the industry. The working fluid is maintained at a suitable pressure adapted to mix easily with the dry particulate sugar.

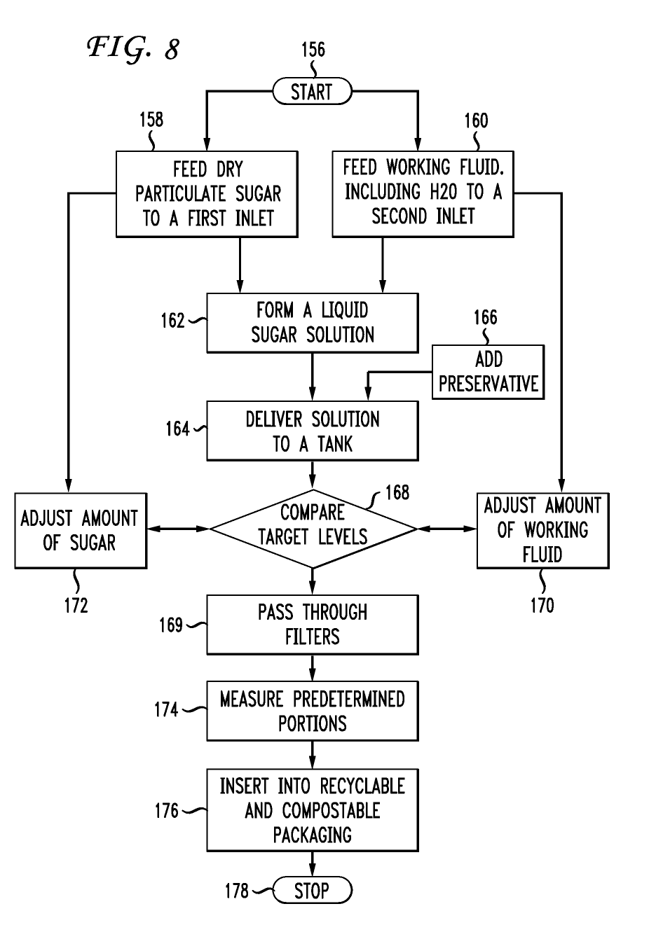

The dry particulate sugar is fed into an eductor-mixer 131 via a first inlet 132 and the working fluid in fed into the educator-mixer 131 by a working fluid circuit via a second inlet 134. The educator-mixer also has a discharge 136 for discharging a liquefied sugar solution into a mixing tank system 137. A heater controller is illustrated at 138 to heat the temperatures to both the working fluid and the sugar to their respective boiling points to facilitate ease of mixing. Specified preservatives from a preservative housing 140 are added via pipes indicated by reference numeral 142. From the mixing tank system 137, the liquefied sugar solution is passed through one or more filters, indicated generally at 139 (see FIGS. 7 and 8), to make sure the product is uniform and free of any radical substances. A measuring device 144 measures the sugar content of the liquefied sugar solution to ensure that target levels of measurements of a brix level, fluidity, and viscosity. The brix level ensures a desired level of sweetness in the concentrated compositions and the fluidity and viscosity levels ensure there is an ideal balance between sweetness levels and ease of dissolution. The ways by which the brix, viscosity, and fluidity can be measured are described at the beginning of the application.

In the event the brix measurement is different from a target sugar content level, a controller system 146 automatically adjusts the amount of sugar supplied to the first inlet 132 of the eductor-mixer 131 and/or the amount of water added to the solution supplied as working fluid to the second inlet 134 of the eductor-mixer Likewise, if the viscosity or fluidity measurements are different from what is desirable, adjustments to the ingredients of the compositions can be made until the desired measurements are accomplished. When the sugar content within the liquefied sugar solution is substantially at the target sugar content level desired, a finished solution outfeed line 148 transfers the finished solution from the tank system 137 to a desired location where individual servings of a predetermined quantity are determined and portioned out. The system components used to carry out this process are indicated by reference numeral 150. From that location, the system operation proceeds to a system component 152 responsible for inserting each individual portion of concentrated liquid sugar into a recyclable and compostable sachet formulated to house each individual serving. The system 125 provides concentrated amounts of liquid sugar on a “continuous” basis to with low equipment costs and low operating temperatures. Moreover, with this system, the sugar concentration of the solution can be selectively varied according to need and which can be automatically adjusted with ease and accuracy.

Referring now to FIG. 8, the process of formulating the concentrated liquid sugar begins operation as indicated by reference numeral 156. At that point, the process continues with the step 158 of dry particulate sugar being either continuously or in batches fed to a first inlet of the eductor-mixer. Concurrently, a working fluid containing water is fed to the second inlet of the eductor-mixer. This step is illustrated by block 160 of the flow diagram illustrated in FIG. 8. The process continues with forming a liquid sugar solution at step 162, which is delivered to the tank at step 164. Preservatives are added at step 166 of the process. The sugar content to water ratio of the solution (brix level) is compared to a desired target at decision block 168. At the same decision block 168, the viscosity and/or fluidity levels of the solution are also determined to ensure that they are at desired levels. In the event it the sugar content to water ratio needs to be adjusted, the controller automatically adjusts either the amount of working fluid at step 170 or the amount of sugar at 172 until the desired target is reached. Once the desired sugar content level is reached, the final solution is passed through one or more filters, indicated at step 169, to ensure uniformity and eliminate any radical substances. The resulting solution is then measured into predetermined portions for individual servings at step 174, which is ultimately packaged into recyclable and compostable sachets for consumer use at step 176. The process ceases at step 178. Alternatively, other processes for formulating the concentrated liquid sugar composition may be used, such as a batch process in which dry sugar is conveyed to a tank of hot liquid (e.g., hot water) and mechanically mixed with the liquid to form a batch of sugar solution. After the batch is finished, the solution is pumped from the tank, usually to a remote location for storage. The process is then repeated to complete the next batch. From the storage location, small individual serving sizes of the solution are determined and packaged in recyclable and compostable sachets for consumer use.

Although the above description may contain specific details, they should not be construed as limiting the claims in any way. Other configurations of the described embodiments of the invention are part of the scope of this invention. For example, the format negotiation may exist on a single compute device and between nodes within that device or the negotiation may occur over a communications network between nodes of the network. Accordingly, the appended claims and their legal equivalents should only define the invention, rather than any specific examples given.

What is claimed is:

1. A liquid sugar composition formulated for individual serving in a recyclable and compostable packaging, comprising:
a ratio of cane sugar and water with a brix level greater than 67.5.

2. A liquid sugar composition as defined in claim 1, further comprising:
one of a predetermined amount of sodium benzoate and citric acid or a predetermined amount of lactic acid.

3. A liquid sugar composition as defined in claim 1, wherein the water is any one of mineral water, natural spring water, filtered water, Artesian water, purified water, distilled water, vapor distilled electrolyte enhanced water, or triple filtered water.

4. A liquid sugar composition as defined in claim 3, further comprising:
one of a predetermined amount of sodium benzoate and citric acid or a predetermined amount of lactic acid.

5. A liquid sugar composition as defined in claim 1, wherein the cane sugar is any one of organic cane sugar, beet sugar, fruit sugar, brown sugar, honey, corn syrup, high fructose corn syrup, and turbinado.

6. A liquid sugar composition as defined in claim 5, further comprising:
one of a predetermined amount of sodium benzoate and citric acid or a predetermined amount of lactic acid.

7. A liquid sugar composition as defined in claim 5, wherein the water is any one of mineral water, natural spring water, filtered water, Artesian water, purified water, distilled water, vapor distilled electrolyte enhanced water, or triple filtered water.

8. A liquid sugar composition as defined in claim 7, further comprising:
a predetermined amount of sodium benzoate and citric acid or a predetermined amount of lactic acid.

9. A liquid sugar substitute composition formulated for individual serving in a recyclable and compostable packaging, further comprising:
a ratio of erythritol and rebiana with water, with a brix level greater than 67.5.

10. A liquid sugar substitute composition as defined in claim 9, further comprising:
one of a predetermined amount of sodium benzoate and citric acid or a predetermined amount of lactic acid.

11. A liquid sugar substitute composition as defined in claim 10, wherein the water is any one of mineral water, natural spring water, filtered water, Artesian water, purified water, distilled water, vapor distilled electrolyte enhanced water, or triple filtered water.

12. A liquid sugar substitute composition as defined in claim 11, further comprising:
a predetermined amount of sodium benzoate and citric acid or a predetermined amount of lactic acid.

14. A liquid sugar substitute composition formulated for individual serving in a recyclable and compostable packaging, further comprising:
a ratio of one of stevia, agave nectar, Brazzein, Curculin, Erythritol, Fructose, Glycyrrhizin, Glycerol, Hydrogenated starch hydrolysates, Inulin, Isomalt, Lactitol, Lo Han Guo, Mabinlin, Maltitol, Maltooligosaccharide, Mannitol, Miraculin, Monatin, Monellin, Pentadin, Sorbitol, Tagatose, Thaumatin, and Xylitol with water, with a brix level greater than 67.5.

15. A liquid sugar substitute composition as defined in claim 14, further comprising:
predetermined amounts of sodium benzoate and citric acid.

16. A liquid sugar substitute composition as defined in claim 14, further comprising:
a predetermined amount of lactic acid.

17. A liquid sugar substitute composition as defined in claim 14, wherein the water is any one of mineral water, natural spring water, filtered water, Artesian water, purified water, distilled water, vapor distilled electrolyte enhanced water, or triple filtered water.

18. A liquid sugar substitute composition as defined in claim 14, further comprising:
one of a predetermined amount of sodium benzoate and citric acid and a predetermined amount of lactic acid.

19. A liquid sugar substitute composition formulated with artificial ingredients for individual serving in a recyclable and compostable packaging, further comprising:
a ratio of water to an artificial ingredient such as one of Saccharine, Aspartame, Sucralose, Dextrose, Isomalt, Mannitol, Trehalone, Sucromalt, Maltitol, Sorbitol, Xylitol, Neotame, Acesulfame-K, Alitame, Cyclamate, Acesulfame Potassium, Alitame, Salt of Aspartame-Acesulfame, Dulcine, Glucin, And Neohesperidin Dihydrochalcone with a predetermined level of sweetness and a viscosity level within a range of 0.080-1.5 Pa·s.

20. A liquid sugar substitute composition as defined in claim 19, further comprising:
one of a predetermined amount of sodium benzoate and citric acid or a predetermined amount of lactic acid.

21. A liquid sugar substitute composition as defined in claim 19, wherein the water is any one of mineral water, natural spring water, filtered water, Artesian water, purified water, distilled water, vapor distilled electrolyte enhanced water, or triple filtered water.

22. A liquid sugar substitute composition as defined in claim 19, further comprising:
one of a predetermined amount of sodium benzoate and citric acid or a predetermined amount of lactic acid.

23. A system for formulating a liquid sugar composition for individual serving in a recyclable and compostable packaging, further comprising:
a sugar feed system for housing and feeding dry particulate sugar content;
a working fluid container for housing and feeding a working fluid comprising water;
a mixer device for blending a predetermined ratio of sugar content with the working fluid to formulate a liquid sugar composition;
a comparator device to compare the Brix level of the liquid sugar composition formulated with a target Brix level;
an adjusting system to adjust the ratio of the sugar content and the working fluid until the target Brix level is reached, thereby resulting in a final concentrated liquid sugar composition;
a measuring system for measuring individual servings of predetermined amounts of the concentrated liquid sugar composition; and
an insertion system for inserting the individual servings of the predetermined amounts of concentrated liquid sugar composition into recyclable and compostable packaging.

24. A system for formulating a liquid sugar composition for individual serving in a recyclable and compostable packaging as defined in claim 23, further comprising:
a mechanism for inserting at least one preservative into the liquid sugar composition.

25. A system for formulating a liquid sugar composition for individual serving in a recyclable and compostable packaging as defined in claim 23, wherein the target Brix degree measurement level is greater than 67.5.

26. A system for formulating a liquid sugar composition for individual serving in a recyclable and compostable packaging as defined in claim 23, wherein target viscosity and fluidity levels are pre-defined and the concentrated liquid sugar solution is measured to meet those target viscosity and fluidity levels.

27. A system for formulating a liquid sugar composition for individual serving in a recyclable and compostable packaging as defined in claim 23, wherein the water is any one of mineral water, natural spring water, filtered water, Artesian water, purified water, distilled water, vapor distilled electrolyte enhanced water, or triple filtered water.

28. A system for formulating a liquid sugar composition for individual serving in a recyclable and compostable packaging as defined in claim 23, wherein the sugar content is any one of cane sugar, organic cane sugar, or erythritol or rebiana.

29. A system for formulating a liquid sugar composition for individual serving in a recyclable and compostable packaging as defined in claim 23, wherein the liquid sugar composition also contains predetermined amounts of either sodium benzoate and citric acid or lactic acid.

30. A system for formulating a liquid sugar composition for individual serving in a recyclable and compostable packaging as defined in claim 23, wherein the recyclable and compostable packing comprises three layers, the first layer of polyethylene terephthalate with polyester, the second layer of an aluminum vacuum metalized version of the first layer, and a third layer to linear low density polyethylene.

31. A method for formulating liquid sugar concentrated compositions for individual serving in a recyclable and compostable packaging, further comprising the steps of:
housing and feeding dry particulate sugar content;
housing and feeding a working fluid comprising water;
blending a predetermined ratio of sugar content with the working fluid to formulate a liquid sugar composition;
comparing a Brix level of the liquid sugar composition formulated with a target Brix level;
adjusting the ratio of the sugar content and the working fluid until the target Brix level is reached, thereby resulting in a final concentrated liquid sugar composition;
measuring individual servings of predetermined amounts of the concentrated liquid sugar composition; and
inserting the individual servings of the predetermined amounts of concentrated liquid sugar composition into a plurality of recyclable and compostable sachets.

32. A method for formulating liquid sugar concentrated compositions for individual serving in a recyclable and compostable packaging as defined in claim 31, further comprising the step of:
inserting at least one preservative into the liquid sugar composition.

33. A method for formulating liquid sugar concentrated compositions for individual serving in a recyclable and compostable packaging as defined in claim 31, wherein the target Brix degree measurement level is greater than 67.5.

34. A method for formulating liquid sugar concentrated compositions for individual serving in a recyclable and compostable packaging as defined in claim 31, wherein the water is any one of mineral water, natural spring water, filtered water, Artesian water, purified water, distilled water, vapor distilled electrolyte enhanced water, or triple filtered water.

35. A method for formulating liquid sugar concentrated compositions for individual serving in a recyclable and compostable packaging as defined in claim 31, wherein the sugar content is any one of cane sugar, organic cane sugar, or erythritol or rebiana.

36. A method for formulating liquid sugar concentrated compositions for individual serving in a recyclable and compostable packaging as defined in claim 31, wherein the target Brix degree measurement level is greater than 67.5.

37. A method for formulating liquid sugar concentrated compositions for individual serving in a recyclable and compostable packaging as defined in claim 31, wherein the liquid sugar composition also contains predetermined amounts of either sodium benzoate and citric acid or lactic acid.

38. A method for formulating liquid sugar concentrated compositions for individual serving in a recyclable and compostable packaging as defined in claim 31, wherein the recyclable and compostable packaging is formulated with three layers, the first layer of polyethylene terephthalate with polyester, the second layer of an aluminum vacuum metalized version of the first layer, and a third layer to linear low density polyethylene.

39. A method for formulating liquid sugar concentrated compositions for individual serving in a recyclable and compostable packaging as defined in claim 31, wherein the recyclable and compostable packaging is configured with a perforated edge to facilitate easy detachment with pressure.

40. A method for formulating liquid sugar compositions for individual serving in a recyclable and compostable packaging as defined in claim 31, wherein the perforated edge when detached uncovers an orifice adapted for easy pouring of the liquid sugar composition.

41. A recyclable and compostable packaging for holding an individual serving of liquid sugar composition, comprising:
a substrate comprising three layers, the first layer of polyethylene terephthalate with polyester;
the second layer of an aluminum vacuum metalized version of the first layer; and
a third layer to linear low density polyethylene.

42. A recyclable and compostable packaging as defined in claim 41, wherein the recyclable and compostable packaging is configured with a perforated edge to facilitate easy detachment with pressure.

43. A recyclable and compostable packaging as defined in claim 41, wherein the perforated edge when detached uncovers an orifice adapted for easy pouring of the liquid sugar composition.

44. A recyclable and compostable packaging as defined in claim 41, further comprising:
a fourth layer for displaying visual indicia relating to the liquid sugar composition for housing within the recyclable and compostable packaging.
